(19) United States
(12) Patent Application Publication
(10) Pub. No.: US 2010/0183530 A1
(43) Pub. Date: Jul. 22, 2010

(54) Cosmetic compositions comprising 4-carboxy-2-pyrrolidinone esters and triazine lipophilic UV-screening agents

(75) Inventors: Hervé Richard, Gagny (FR); Benoit Muller, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/654,033

(22) Filed: Dec. 8, 2009

Related U.S. Application Data
(60) Provisional application No. 61/193,649, filed on Dec. 12, 2008.

(30) Foreign Application Priority Data
Dec. 8, 2008 (FR) 0858339

(51) Int. Cl. A61K 8/49; A61Q 17/04; A61K 8/58

(52) U.S. Cl. 424/60; 424/59

(57) ABSTRACT

UV-screening compositions, formulated into a cosmetically acceptable medium, contain at least one ester solvent derived from 4-carboxy-2-pyrrolidinone, of formula (I) below:

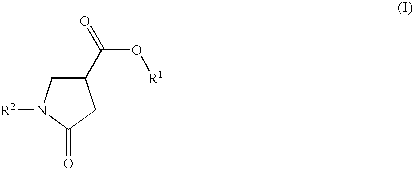

in which:
R¹ is a linear or branched C₁-C₂₀ alkyl radical,
R² is a linear or branched C₁-C₂₀ alkyl radical that may contain a C₅-C₆ ring member, the phenyl radical, the benzyl radical or the phenethyl radical, and at least one triazine-type lipophilic UV-screening agent.

### CROSS-REFERENCE TO PRIORITY/PROVISIONAL APPLICATIONS

This application claims priority under 35 U.S.C. §119 of FR 0858339, filed Dec. 8, 2008 and §120 of U.S. Provisional Application No. 61/193,649, filed Dec. 12, 2008, each hereby expressly incorporated by reference and each assigned to the assignee hereof.

### BACKGROUND OF THE INVENTION

1. Technical Field of the Invention

The present invention relates to compositions comprising, formulated into a cosmetically acceptable medium, at least one ester derived from 4-carboxy-2-pyrrolidinone, of formula (I), the definition of which is given hereinafter, and at least one triazine lipophilic UV-screening agent.

The present invention also relates to the formulation of at least one ester derived from 4-carboxy-2-pyrrolidinone, of formula (I), the definition of which is given hereinafter, into compositions comprising, in a cosmetically acceptable medium, at least one triazine lipophilic UV-screening agent, as a solvent for said triazine screening agent and/or as an agent for improving the solubility of said triazine UV-screening agent in said composition.

2. Description of Background and/or Related and/or Prior Art

Many cosmetic or dermatological products are provided in various galenical forms comprising a liquid fatty phase, such as dispersions, oily lotions, oil/water, water/oil or multiple emulsions, or cream gels. Certain particularly advantageous cosmetic or dermatological active agents, such as lipophilic organic screening agents, are not readily soluble in the oily phase of these formulations and have a tendency during storage to form crystals or to precipitate, in particular in emulsions. Such phenomena are undesirable from the point of view of stability of the formulation and/or with respect to consumer comfort insofar as they can destabilize the composition and/or affect the aesthetic appearance of the product and/or lead to cosmetic discomfort upon application to the skin and/or the hair, or else to the concentration of active agents being limited in these formulations, which means that products that are sufficiently effective cannot be obtained.

In particular, anti-sun (sunscreen) compositions are often in the form of an oil-in-water or water-in-oil emulsion, of gels or of anhydrous products which contain, at various concentrations, one or more lipophilic and/or hydrophilic, insoluble and/or soluble, organic and/or inorganic screening agents capable of selectively absorbing harmful UV-radiation. These screening agents and the amounts thereof are selected according to the desired protection factor. Depending on whether they are lipophilic or, conversely, hydrophilic in nature, these screening agents can be distributed, respectively, either into the fatty phase or into the aqueous phase of the final composition.

UV-screening agents of the triazine type are lipophilic UV-B screening agents that are particularly advantageous for anti-sun cosmetic formulations. Certain of these can screen out UV-A radiation. However, their photoprotective capacity when formulated is quite limited in the usual cosmetic carriers containing oils, such as oxyethylenated or oxypropylenated fatty (mono/poly) alcohols (“Cetiol HE” from Henkel or “Witconol APM” from Witco) or else fatty esters such as C₁₂-C₁₅ alkyl benzoate (“Finsolv TN” from Finetex), fatty acid triglycerides, for example Miglyol® 812 marketed by Dynamit Nobel, or amino acid derivatives (“Eldew SL205” from Ajinomoto), because the solubility of these screening agents in these oils commonly used in formulation is not completely satisfactory. The result of this is: either the appearance over time of crystallization in the formulations, which is detrimental to good quality, stability and effectiveness of the anti-sun products; or the fact that the concentration of screening agents in the formulations has to be limited, thereby making it impossible to obtain sufficiently effective products.

Thus, there exists need to develop novel solvents for effectively dissolving lipophilic screening agents of the triazine type to improve their solubility in the oils in the cosmetic or dermatological formulation carriers containing them, without the drawbacks noted above.

Pyrrolidone derivatives are known as penetrating agents for active agents such as oleocanthal in WO 2008/01240 or such as asprotadil alkyl esters in U.S. Pat. No. 6,673,841.

### SUMMARY OF THE INVENTION

Surprisingly, a novel family of effective solvents has now been developed, constituted of esters derived from 4-carboxy-2-pyrrolidinone, of formula (I), the definition of which is given hereinafter, which make it possible to achieve the above objective. These compounds can be incorporated into many cosmetic products.

This discovery provides the basis of the present invention.

The present invention features, in particular, compositions comprising, formulated into a cosmetically acceptable medium, at least one ester derived from 4-carboxy-2-pyrrolidinone, of formula (I), and at least one triazine-type UV-screening agent.

The present invention features formulating at least one ester derived from 4-carboxy-2-pyrrolidinone, of formula (I), the definition of which is given hereinafter, into compositions comprising, in a cosmetically acceptable medium, at least one triazine-type UV-screening agent, as a solvent for said active agent and/or as an agent for improving the solubility of said triazine-type UV-screening agent in said compositions.

The present invention also features topical application onto the skin of at least one diester derived from pyrrolidinone, of formula (I), formulated in a composition comprising, in a cosmetically acceptable medium, at least one triazine-type lipophilic UV-screening agent, for improving the sun protection factor of such UV-screening agents.

Other characteristics, aspects and advantages of the invention will become apparent from the detailed description which follows.

The term “cosmetically acceptable” means compatible with the skin and/or its appendages, which has a pleasant color, odor and feel and which does not create any unacceptable discomfort (tingling, taughtness, redness) that may dissuade the consumer from using this composition.

The term “lipophilic screening agent” means any cosmetic or dermatological organic UV-screening agent capable of being completely dissolved in the molecular state in a liquid fatty phase or else of being solubilized in colloidal form (for example in micellar form) in a liquid fatty phase.

### DETAILED DESCRIPTION OF BEST MODE AND SPECIFIC/PREFERRED EMBODIMENTS OF THE INVENTION

The esters derived from 4-carboxy-2-pyrrolidinone in accordance with the invention are selected from among those corresponding to general formula (I) below:

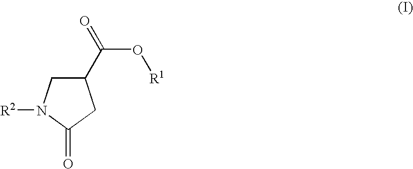

in which:

R¹ is a linear or branched C₁-C₂₀ alkyl radical, and

R² is a linear or branched C₁-C₂₀ alkyl radical optionally comprising a C₅-C₆ ring member, the phenyl radical, the benzyl radical or the phenethyl radical.

In formula (I), exemplary alkyl radicals particularly include the methyl, ethyl, n-propyl, isopropyl, n-butyl, isobutyl, tert-butyl, n-octyl, 2-ethylhexyl, dodecyl, hexadecyl, cyclohexyl and methylcyclohexyl radicals.

Among the compounds of formula (I), particularly preferred are the following compounds (a) to (oo):

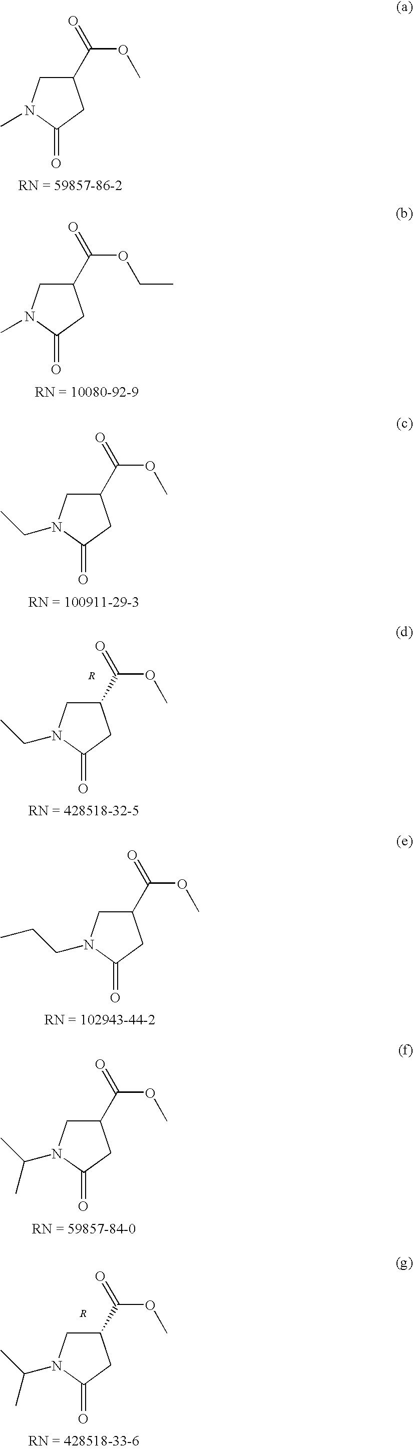

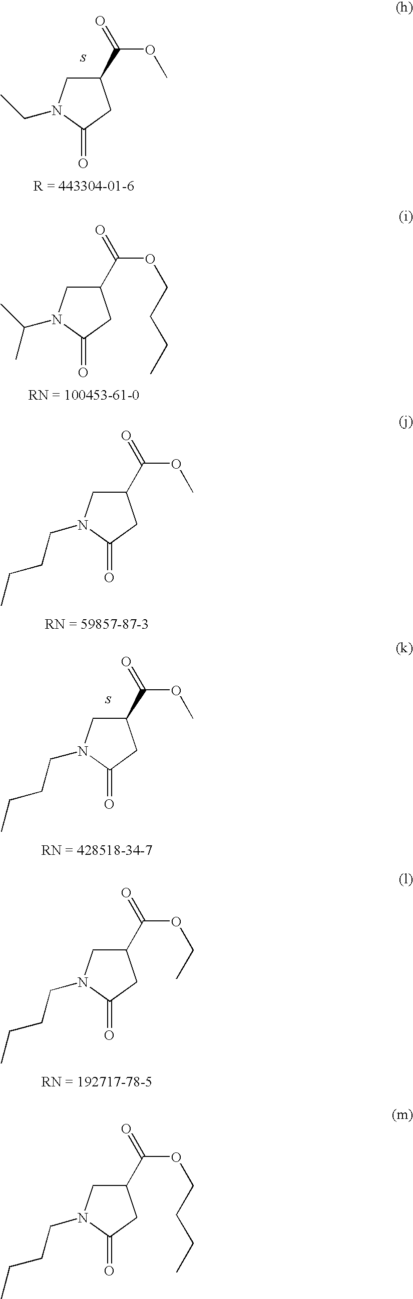

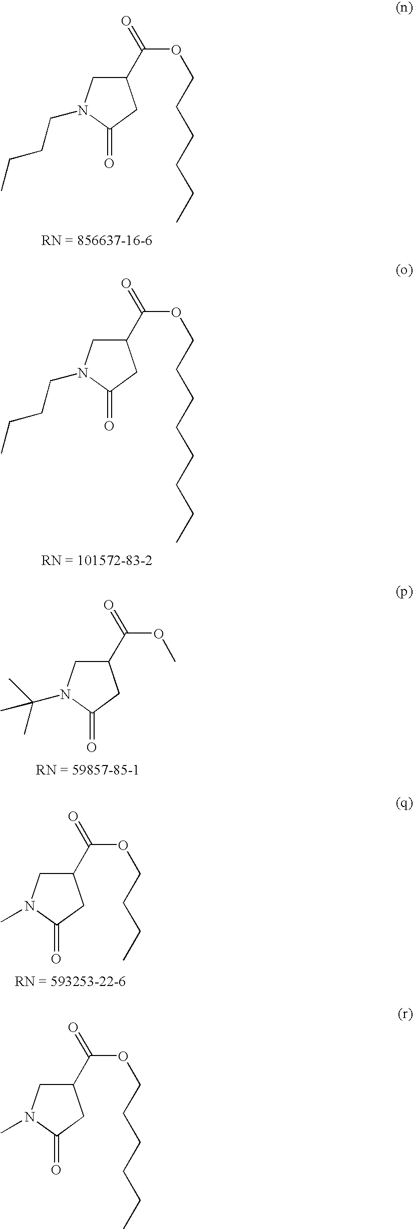

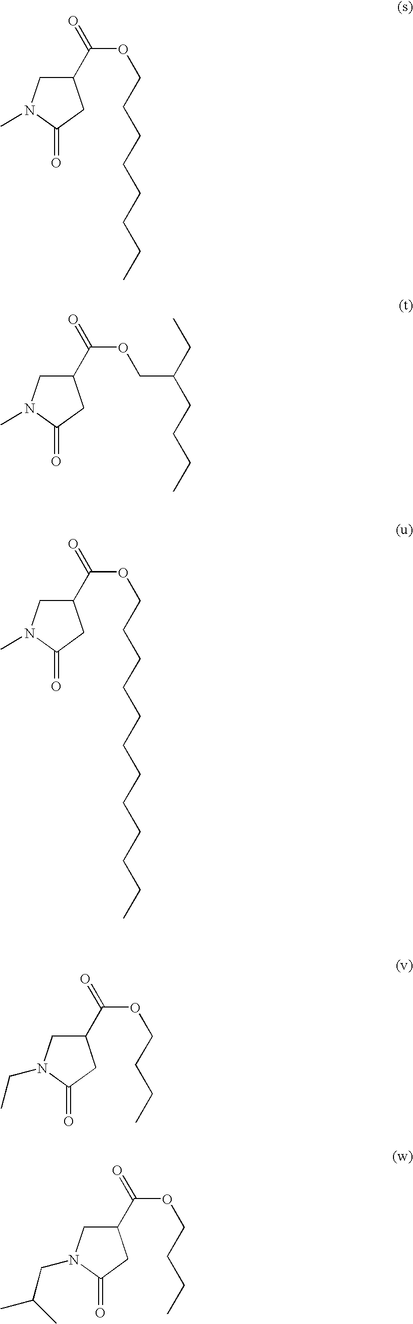

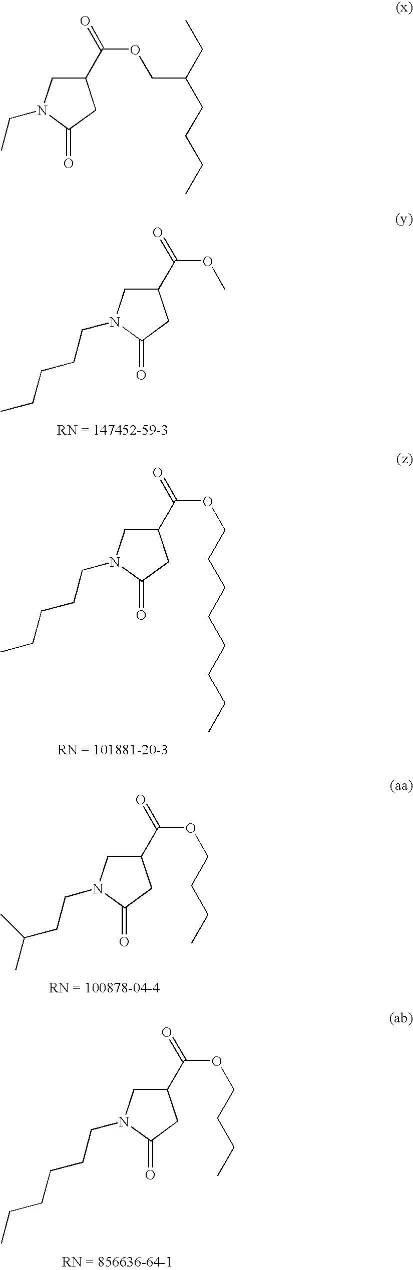

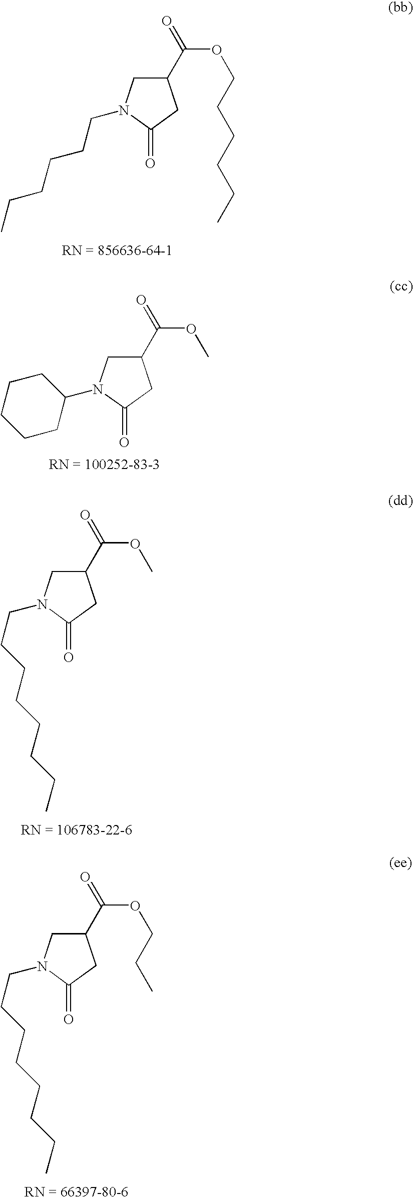

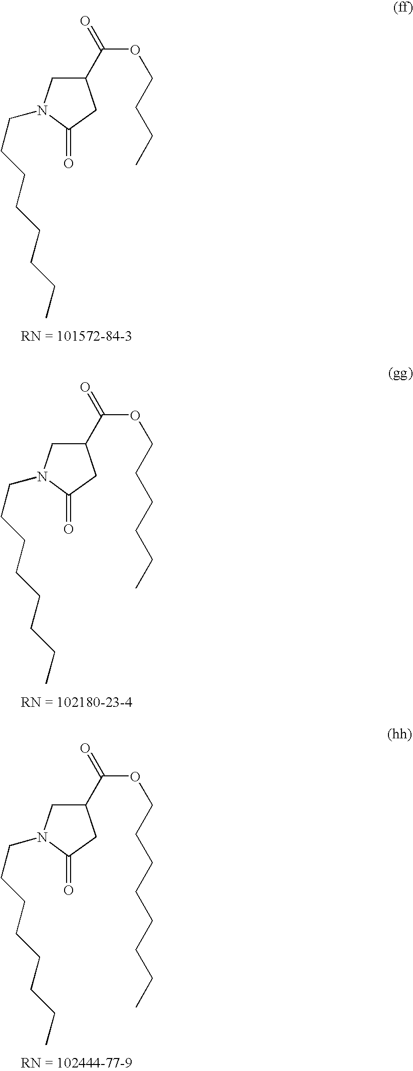

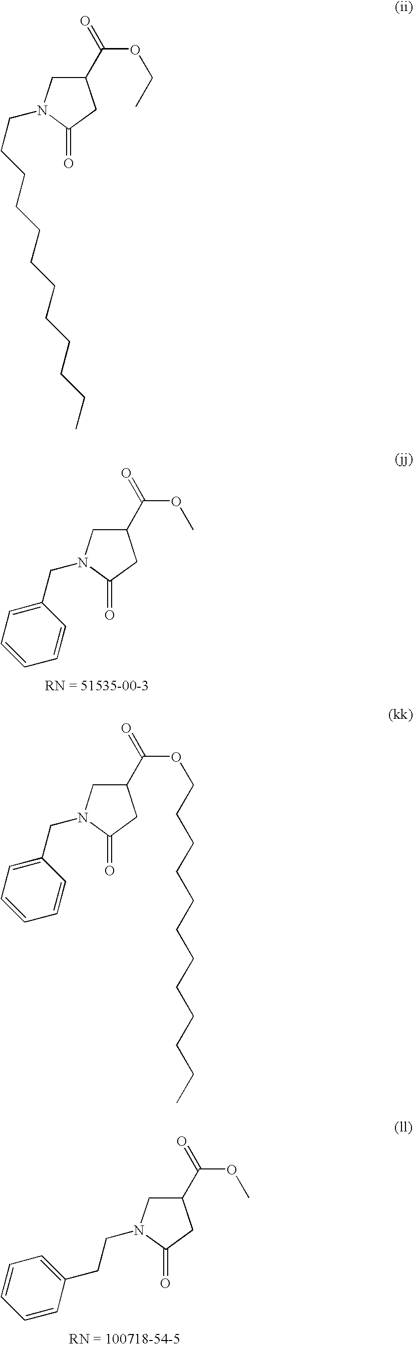

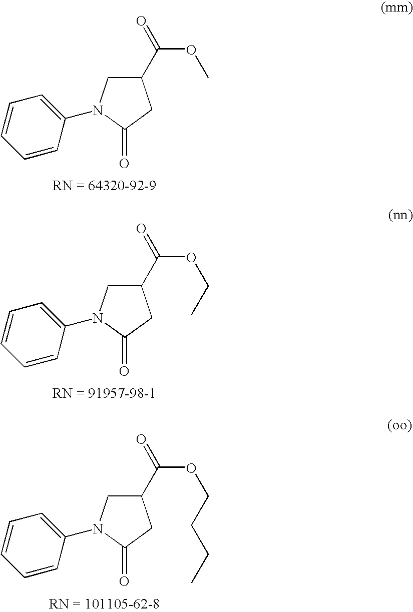

More particularly preferred are the derivatives of examples (t), (u), (v), (x), (j), (l), (m), (w), (n), (ab), (ii) and (kk), and even more particularly the compounds (j), (l) and (m).

The compounds of formula (I), the syntheses of which are described in the following articles: J. Org. Chem., 26, pages 1519-24 (1961); Tetrahedron Asymmetric, 12 (23), pages 3241-9 (2001); J. Industrial & Engineering Chem., 47, pages 1572-8 (1955); J. Am. Chem. Soc., 60, pages 402-6 (1938); and in EP-0069512, U.S. Pat. Nos. 2,811,496 (1955), 2,826,588, 3,136,620, FR-2290199 and FR-2696744, are readily prepared:

either by condensation of a diester of itaconic acid of formula (II) with a primary amine of formula (III), with or without solvent at a temperature from 20° C. to 150° C. according to the following reaction scheme:

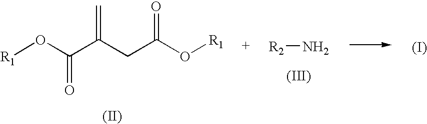

or in 2 stages starting from the itaconic acid of formula (IV) by condensation with the primary amine of formula (III), in the presence or absence of a solvent, so as to give the intermediate acid of formula (V), followed by esterification of this acid of formula (V) in the presence of an excess of alcohol of formula (VI) according to the following reaction scheme:

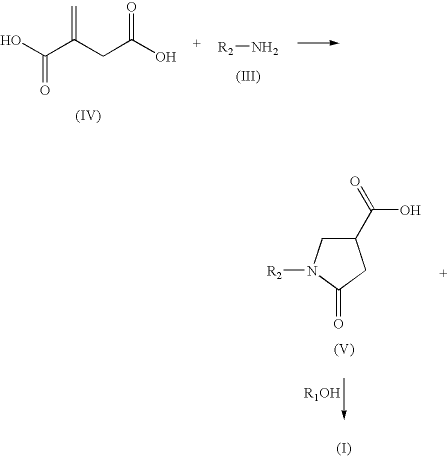

or the derivatives of formula (I) comprising an ester chain R′₁ (linear or branched C₃-C₂₀ alkyl radical) can also be obtained by transesterification of the derivatives comprising an ester chain R₁ (methyl or ethyl radical) in the presence of a linear or branched C₃-C₂₀ alcohol and of a tin catalyst or titanium catalyst according to the following reaction scheme:

Preferably, the derivative(s) of formula (I) in accordance with the invention is (are) present in the compositions of the invention at contents ranging from 1% to 30% by weight, and more preferably from 3% to 20% by weight, relative to the total weight of the composition.

According to one particular embodiment of the invention, the derivative(s) of formula (I) in accordance with the invention is (are) present in an amount sufficient to solubilize, by itself or by themselves (without it being necessary to use another solvent) the entire amount of lipophilic triazine screening agent(s) present in the composition.

The triazine-type UV-screening agents in accordance with the invention may be in particular selected from among 1,3,5-triazine derivatives of the formula (1) below:

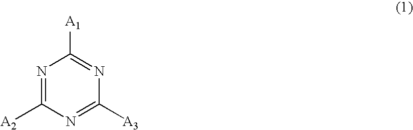

in which the A₁, A₂ and A₃ radicals, which may be identical or different, are selected from among groups of formula (2):

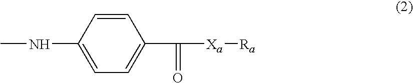

in which:

the bridges Xₐ, which may be identical or different, are each oxygen or an —NH— radical;

the radicals Rₐ, which may be identical or different, are each selected from among: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylenated radical comprising from 1 to 6 ethylene oxide units and the terminal OH group of which is methylated; a radical of formula (3), (4) or (5) below:

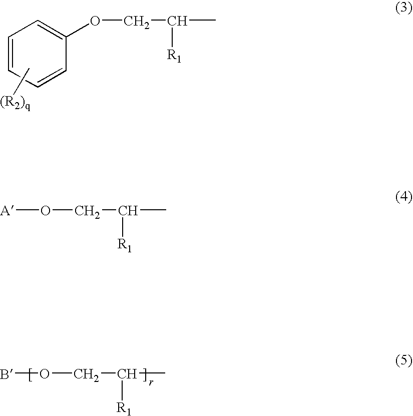

in which:

R₁ is hydrogen or a methyl radical;

R₂ is a C₁-C₉ alkyl radical;

q is an integer ranging from 0 to 3;

r is an integer ranging from 1 to 10;

A′ is a C₄-C₈ alkyl radical or a C₅-C₈ cycloalkyl radical;

B′ is selected from among: a linear or branched C₁-C₈ alkyl radical; a C₅-C₈ cycloalkyl radical; and an aryl radical optionally substituted with one or more C₁-C₄ alkyl radicals.

A first more particularly preferred family of 1,3,5-triazine derivatives, in particular described in EP-A-0,517,104, is that of the 1,3,5-triazines corresponding to formula (1) in which A₁, A₂ and A₃ are of formula (2) and have the following characteristics:

one of the Xₐ—Rₐ radicals is the —NH—Rₐ radical with Rₐ selected from among: a C₅-C₁₂ cycloalkyl radical optionally substituted with one or more C₁-C₄ alkyl radicals; a radical of formula (3), (4) or (5) above in which:

B′ is a C₁-C₄ alkyl radical;

R₂ is the methyl radical;

the other two Xₐ—Rₐ represent the —O —Rₐ radical with Rₐ, which may be identical or different, selected from among: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; and a radical of formula (3), (4) or (5) above in which:

B′ is a C₁-C₄ alkyl radical;

R₂ is the methyl radical.

A second more particularly preferred family of 1,3,5-triazine derivatives, in particular described in EP-A-0,570,838, is that of the 1,3,5-triazines corresponding to formula (I) in which A₁, A₂ and A₃ are of formula (2) and having all the following characteristics:

one or two Xₐ—Rₐ represent(s) the —NH—Rₐ radical, with Rₐ selected from among: a linear or branched C₁-C₁₈ alkyl radical; a C₅-C₁₂ cycloalkyl radical optionally substituted with one or more C₁-C₄ alkyl radicals; and a radical of formula (3), (4) or (5) above in which:

B′ is a C₁-C₄ alkyl radical;

R₂ is the methyl radical;

the or the two other Xₐ—Rₐ being the —O—Rₐ radical with Rₐ, which may be identical or different, selected from among: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; and a radical of formula (3), (4) or (5) above in which:

B′ is a C₁-C₄ alkyl radical;

R₂ is the methyl radical.

A particularly preferred 1,3,5-triazine of this second family is 2-[(p-(tert-butylamido)anilino]-4,6-bis[(p-(2′-ethylhexyl-1′-oxycarbonyl)anilino]-1,3,5-triazine or “diethylhexyl butamido triazone” marketed under the trademark “Uvasorb HEB” by Sigma 3V and corresponding to the formula below:

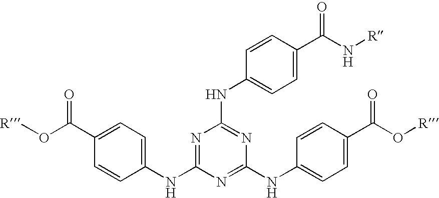

in which R′″ is a 2-ethylhexyl radical and R″ is a tert-butyl radical.

A third preferred family of compounds of the present invention, and which is in particular described in U.S. Pat. No. 4,724,137, is that of the 1,3,5-triazines corresponding to formula (I) in which A₁, A₂ and A₃ are of formula (2) and have the following characteristics:

Xₐ are identical and represent oxygen;

Rₐ, which may be identical or different, are each a C₆-C₁₂ alkyl radical or a polyoxyethylenated radical comprising from 1 to 6 ethylene oxide units and the terminal OH group of which is methylated.

A particularly preferred 1,3,5-triazine of this third family is 2,4,6-tris[p-(2′-ethylhexyl-1′-oxycarbonyl)anilino]-1,3,5-triazine or “ethylhexyl triazone” marketed in particular under the trademark “Uvinul T 150” by BASF, and corresponds to the formula below:

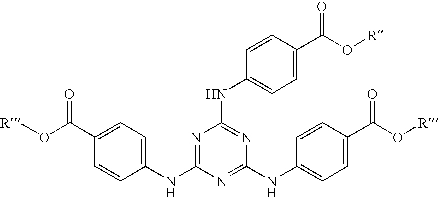

in which R′″ is a 2-ethylhexyl radical.

The triazine-type UV-screening agents may also be selected from among bis(resorcinyl)triazine derivatives in accordance with the present invention corresponding to formula (6) below:

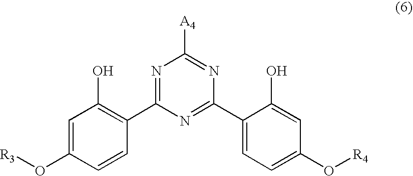

in which:

the R₃ and R₄ radicals, which may be identical or different, are each a C₃-C₁₈ alkyl radical, a C₂-C₁₈ alkenyl radical or a residue of formula —CH₂—CH(OH)—CH₂—OT₁ where T₁ is a hydrogen atom or a C₁-C₈ alkyl radical;

A₄ is a residue corresponding to one of the formulae below:

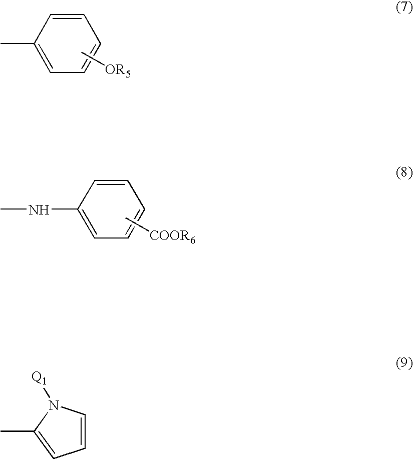

in which:

R₅ is a hydrogen atom, a C₁-C₁₀ alkyl radical, a radical of formula: —(CH₂CHR₇—O)ₙ₁R₆ where n₁ is a number from 1 to 16, or else a residue having the structure —CH₂—CH—(OH)—CH₂OT₁ with T₁ having the same meaning indicated above;

R₆ is hydrogen, a metal cation M, a C₁-C₅ alkyl radical or a residue of formula —(CH₂)ₘ₂—OTᵢ where m₂ is a number from 1 to 4 and T₁ has the same meaning indicated above;

R₇ is hydrogen or methyl;

Q₁ is a C₁-C₁₈ alkyl radical.

In formulae (6) to (9) described above:

the alkyl radicals are linear or branched and may be selected, for example, from among methyl, ethyl, n-propyl, isopropyl, n-butyl, sec-butyl, tert-butyl, amyl, isoamyl, tert-amyl, heptyl, octyl, isooctyl, nonyl, decyl, undecyl, dodecyl, tetradecyl, pentadecyl, hexadecyl, heptadecyl or octadecyl;

the alkenyl radicals may be selected, for example, from among allyl, methallyl, isopropenyl, 2-butenyl, 3-butenyl, isobutenyl, n-penta-2,4-dienyl, 3-methylbut-2-enyl, n-2-octenyl, n-2-dodecenyl, isododecenyl and n-4-octadecenyl;

the alkoxy radicals are linear or branched and may be selected, for example, from among methoxy, ethoxy, n-propoxy, isopropoxy, n-butoxy, sec-butoxy, tert-butoxy, amyloxy, isoamyloxy or tert-amyloxy;

the C₁-C₅ monoalkylamino or dialkylamino radicals may be selected, for example, from among methylamino, ethylamino, propylamino, n-butylamino, sec-butylamino, tert-butylamino, pentylamino, dimethylamino, diethylamino, dibutylamino or methylethylamino;

the metal cations are alkali metal, alkaline-earth metal or metal cations selected, for example, from lithium, potassium, sodium, calcium, magnesium, copper and zinc.

The bis(resorcinyl)triazine derivatives of formula (6) of the invention are screening agents that are already known per se. They are described and prepared according to the syntheses indicated in EP-A-0,775,698.

By way of examples of compounds of formula (6) that can be formulated, exemplary are:
2,4-bis{[4-(2-ethylhexyloxy)-2-hydroxy]phenyl}-6-(4-methoxyphenyl)-1,3,5-triazine;
2,4-bis{[4-(3-(2-propyloxy)-2-hydroxypropyloxy)-2-hydroxyl]phenyl}-6-(4-methoxyphenyl)-1,3,5-triazine;
2,4-bis{[4-(2-ethylhexyloxy)-2-hydroxy]phenyl}-6-[4-(2-methoxyethylcarboxyl)phenylamino]-1,3,5-triazine,
2,4-bis{[4-(2″-methylpropenyloxy)-2-hydroxy]phenyl}-6-(4-methoxyphenyl)-1,3,5-triazine;
2,4-bis{[4-(3-(2-propyloxy)-2-hydroxypropyloxy]-2-hydroxy]phenyl}-6-[(4-ethylcarboxyl)phenylamino]-1,3,5-triazine;
2,4-bis{[4-(2-ethylhexyloxy)-2-hydroxy]phenyl}-6-(1-methylpyrrol-2-yl)-1,3,5-triazine.

The compound derived from bis(resorcinyl)triazine that is more particularly preferred according to the invention will be the compound 2,4-bis{[4-(2-ethylhexyloxy)-2-hydroxy]phenyl}-6-(4-methoxyphenyl)-1,3,5-triazine (INCI name: Bis-Ethylhexyloxyphenol Methoxyphenyl Triazine) marketed under the trademark “Tinosorb S” by Ciba Geigy.

Exemplary esters of benzalmalonate with s-triazine, as described in EP507691, EP507692, EP790243 and EP944624, are in particular the following compounds:
2,4,6-tris(diisobutyl 4′-aminobenzalmalonate)-6-butoxy-s-triazine,
2,4,6-tris(diisobutyl 4′-aminobenzalmalonate)-6-(2-ethylhexylamino)-s-triazine,
2,4-bis(4′-aminobenzylidenecamphor)-6-(2-ethylhexylamino)-s-triazine,
2,4,6-bis(4′-aminobenzylidenecamphor)-6-(diisobutyl 4′-aminobenzalmalonate)-s-triazine,
2,4,6-tris(diethyl 4′-aminobenzalmalonate)-s-triazine,
2,4,6-tris(diisopropyl 4′-aminobenzalmalonate)-s-triazine,
   and more particularly 2,4,6-tris(diisobutyl 4′-aminobenzalmalonate)-s-triazine.

The triazine-type UV-screening agents may also be selected from among those described in EP 0841341, EP 1471059, EP 1568700, EP1642893 and EP 1815884.

Exemplary are:
2,4,6-tris(dineopentyl 4′-aminobenzalmalonate)-s-triazine,
2,4-bis(n-butyl 4′-aminobenzoate)-6-(aminopropyltrisiloxane)-s-triazine.

The triazine-type UV-screening agents may be in particular selected from among those corresponding to general formula (9) below, or one of the tautomeric forms thereof

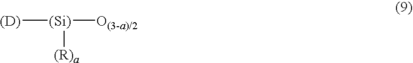

in which:

the radicals R, which may be identical or different, are each a linear or branched and optionally halogenated or unsaturated C₁-C₃₀ alkyl radical, a C₆-C₁₂ aryl radical, a C₁-C₁₀ alkoxy radical, a hydroxyl radical or the trimethylsilyloxy radical;

a=1 to 3; in addition to the structural units of formula -A-(Si)(R)ₐ(O)_{(3-a)/2}.

The organosiloxane may comprise structural units of formula: (R)_{b}—(Si)(O)_{(4-b)/2} in which R has the same definition in formula (9), b=1, 2 or 3;

the group (D) is an s-triazine compound of formula (10) below:

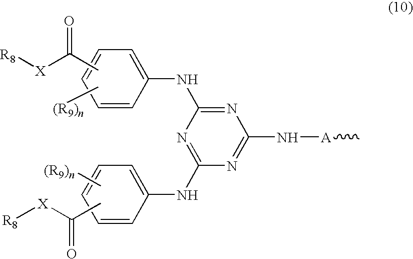

wherein:

X is —O— or —NR₁₀—, with R₁₀ representing hydrogen or a C₁-C₅ alkyl radical,

R₈ is a linear or branched, optionally unsaturated, C₁-C₃₀ alkyl radical that may contain a silicon atom, a C₅-C₂₀ cycloalkyl group optionally substituted with 1 to 3 linear or branched C₁-C₄ alkyl radicals, the —(CH₂CHR₁₀—O)ₘR₁₁ group or the —CH₂—CH(OH)—CH₂—O—R₁₂ group,

the radicals R₉, which may be identical or different, are each a hydroxyl radical, a linear or branched C₁-C₈ alkyl radical, or a C₁-C₈ alkoxy radical, with the proviso that two adjacent R₂ of the same aromatic ring may together form an alkylidene dioxy group in which the alkylidene radical contains 1 or 2 carbon atoms,

R₁₀ is hydrogen or methyl; with the proviso that the (C═O)XR₈ group to be in the ortho-, meta- or para-position with respect to the amino group,

R₁₁ is hydrogen or a C₁-C₈ alkyl group,

R₁₂ is hydrogen or a C₄-C₈ alkyl group,

m is an integer ranging from 2 to 20,

n=0 to 2,

A is a divalent radical selected from among methylene or a group corresponding to one of formula (11), (12), (13) or (14) below:

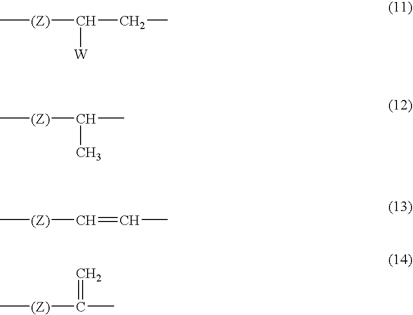

in which:

Z is a saturated or unsaturated, linear or branched C₁-C₁₀ alkylene diradical optionally substituted with a hydroxyl radical or oxygens and that may optionally contain an amino group, and

W is a hydrogen atom, a hydroxyl radical or a saturated or unsaturated, linear or branched C₁-C₈ alkyl radical.

It should be noted that the derivatives of formula (9) can be formulated in their tautomeric forms, and more particularly in the tautomeric form of formula (9′) below:

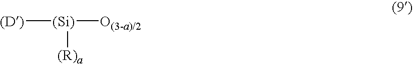

in which the (D′) group is an s-triazine compound of formula (10′) below:

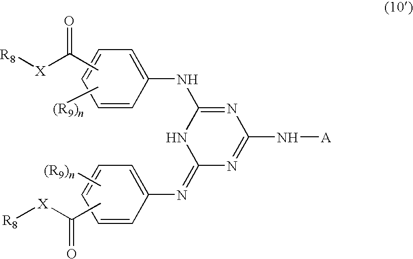

In addition to the units of formula -A-(Si)(R)ₐ(O)_{(3-a)/2}, the organosiloxane may comprise units of formula (R)_{b}—(Si)(O)_{(4-b)/2} in which R has the same definition as in formula (9), b=1, 2 or 3.

In formulae (9) and (9′) as defined above, the alkyl radicals may be linear or branched, saturated or unsaturated, and selected in particular from among methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The alkyl radical particularly preferred is the methyl radical.

The preferred s-triazine derivatives are those for which, in formula (9) or (9′), at least one, and even more preferably all, of the following characteristics are satisfied:

R is methyl,

a=1 or 2,

R₈ is a C₂-C₈ radical,

Z=—CH₂—,

W═H.

Preferably, the s-triazine compounds of formula (9) of the invention are represented by formula (9a), (9b) or (9c) below:

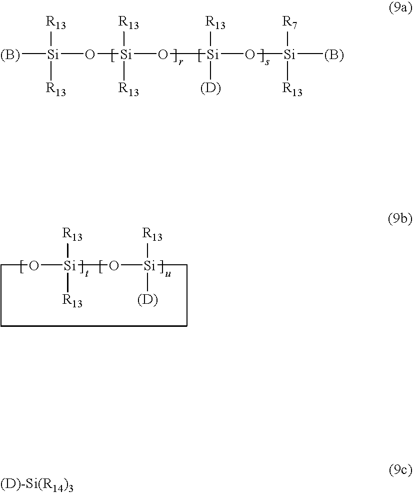

in which:

(D) corresponds to formula (10) as defined above,

the radicals R₁₃, which may be identical or different, are selected from among linear or branched C₁-C₂₀ alkyl, phenyl, 3,3,3-trifluoropropyl and trimethylsilyloxy radicals or the hydroxyl radical,

the radicals R₁₄, which may be identical or different, are selected from among linear or branched C₁-C₂₀ alkyl and alkenyl radicals, and hydroxyl or phenyl radicals,

(B), which may be identical or different, are selected from among R₁₃ radicals and the (D) radical,

r is an integer ranging from 0 to 200, inclusive,

s is an integer ranging from 0 to 50 and, if s=0, at least one of the two symbols (B) is (D),

u is an integer ranging from 1 to 10,

t is an integer ranging from 0 to 10, with the proviso that t+u is greater than or equal to 3, and also the tautomeric forms thereof.

The linear diorganosiloxanes of formula (9a) are particularly preferred.

The linear or cyclic diorganosiloxanes of formula (9a) or (9b) according to the present invention are random oligomers or polymers preferably having at least one, and even more preferably all, of the following characteristics:

R₁₃ is the methyl radical, the C₁-C₂ alkoxy radical or the hydroxyl radical,

B is preferably methyl (the case of the linear compounds of formula (9a)).

Particularly preferred compounds of formula (9) are the following compounds of formulae (9₁) to (9₁₄), and also the tautomeric forms thereof:

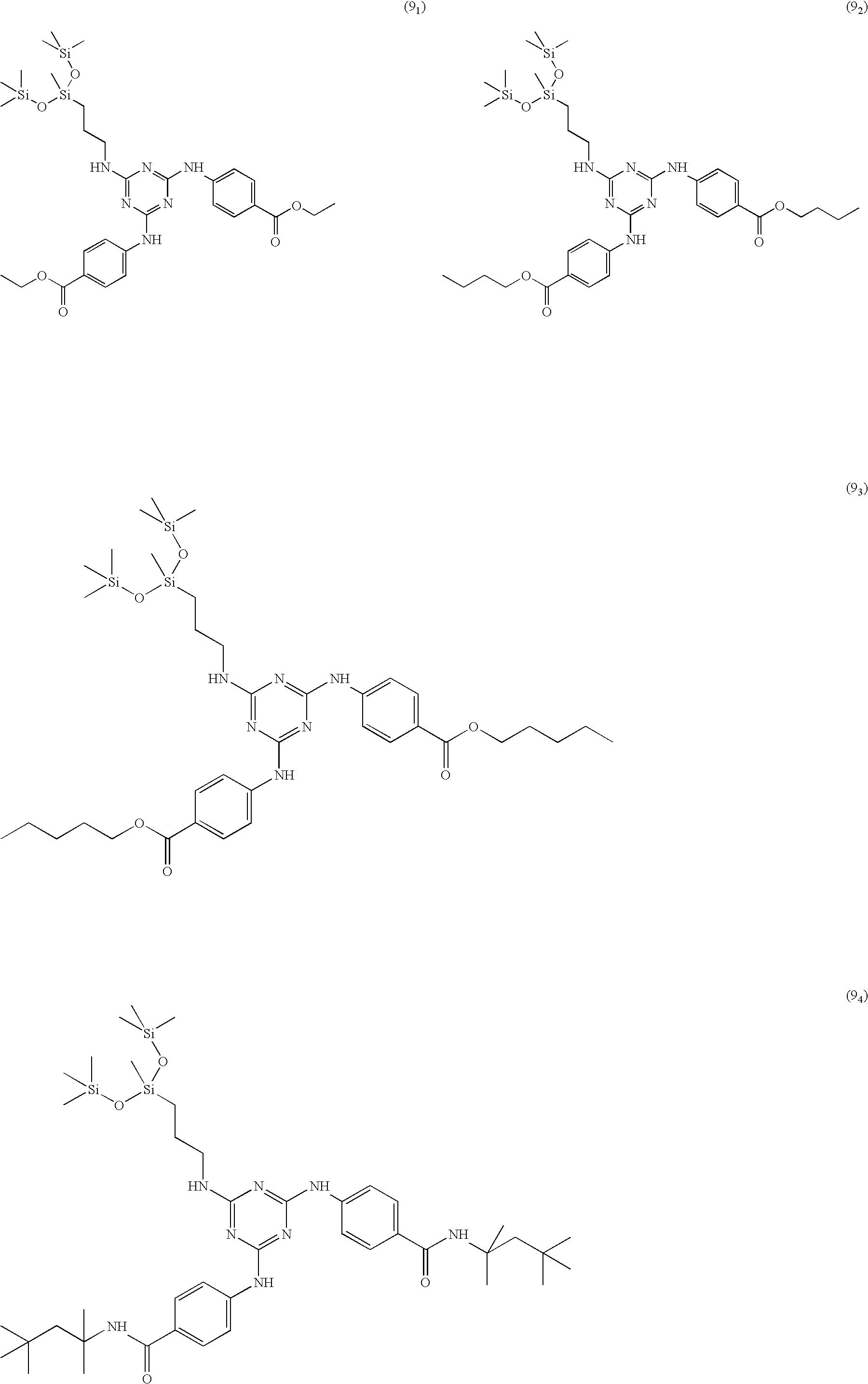

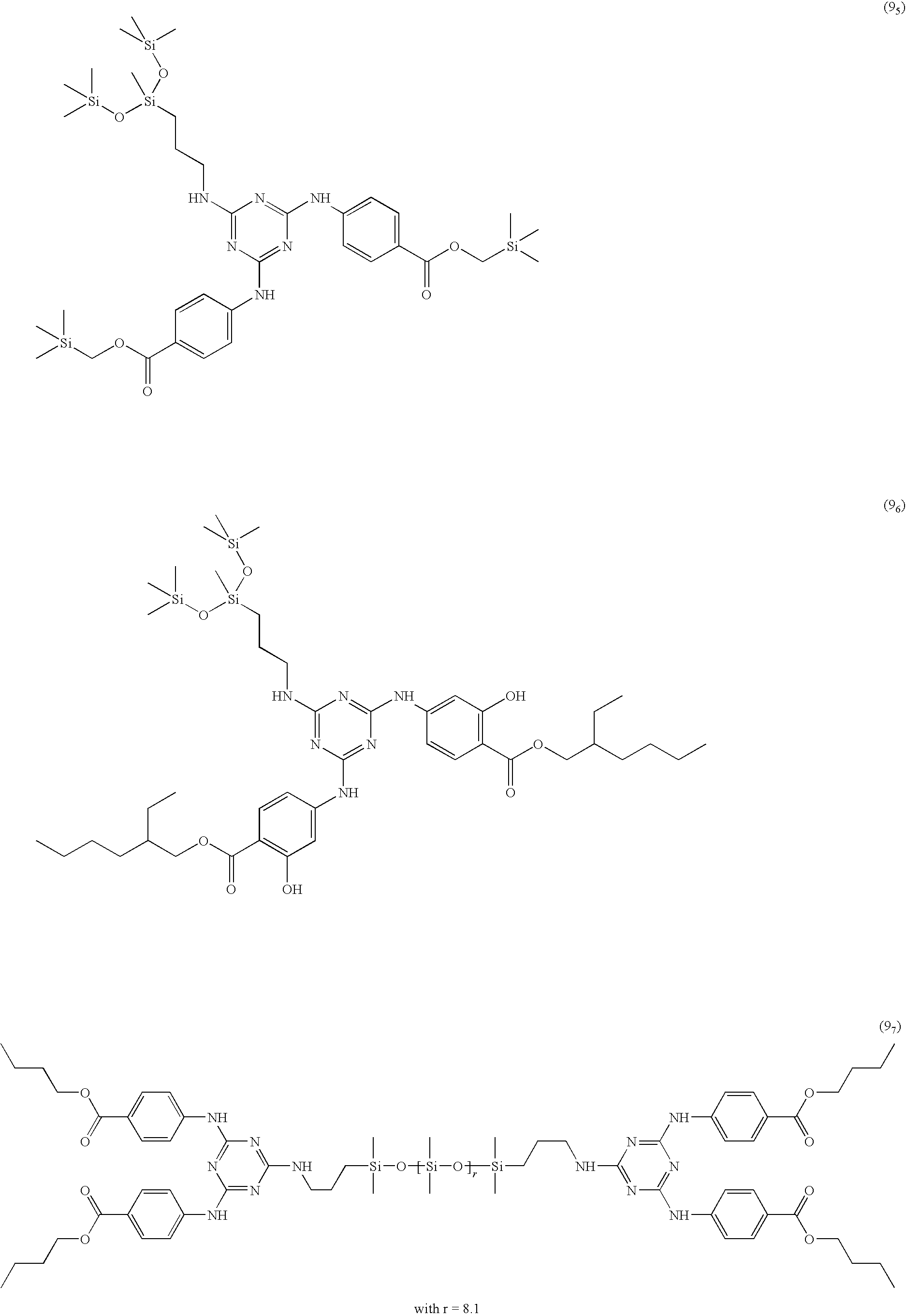

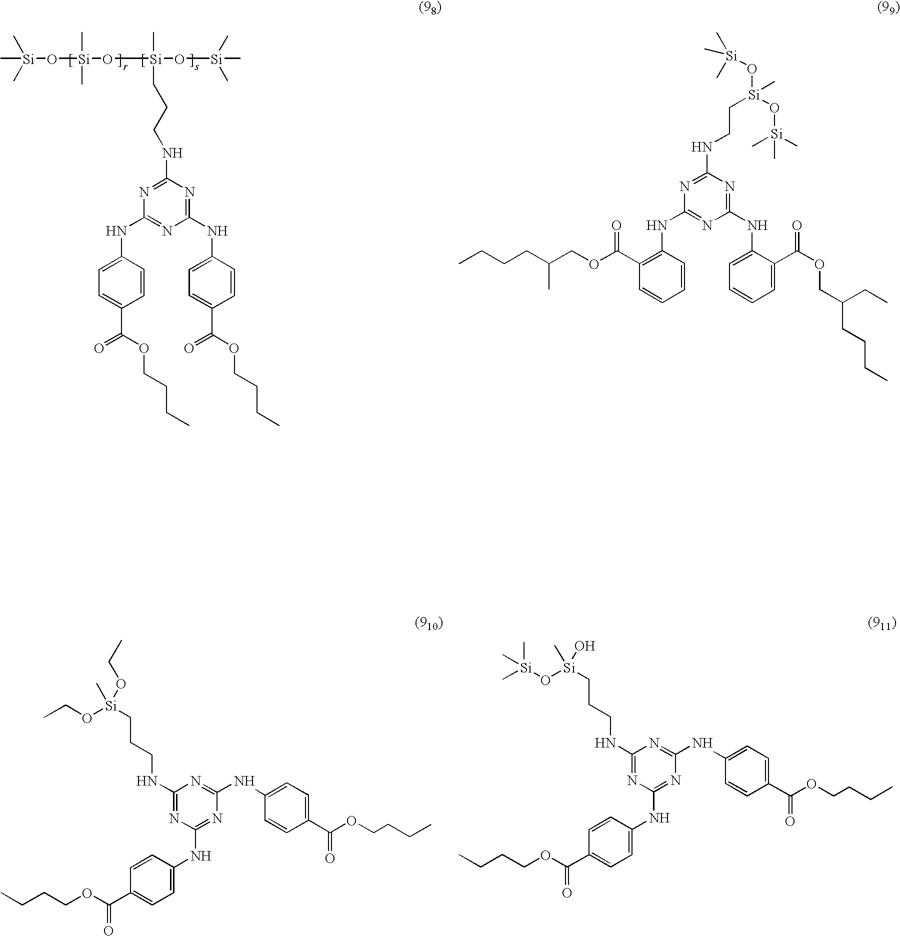

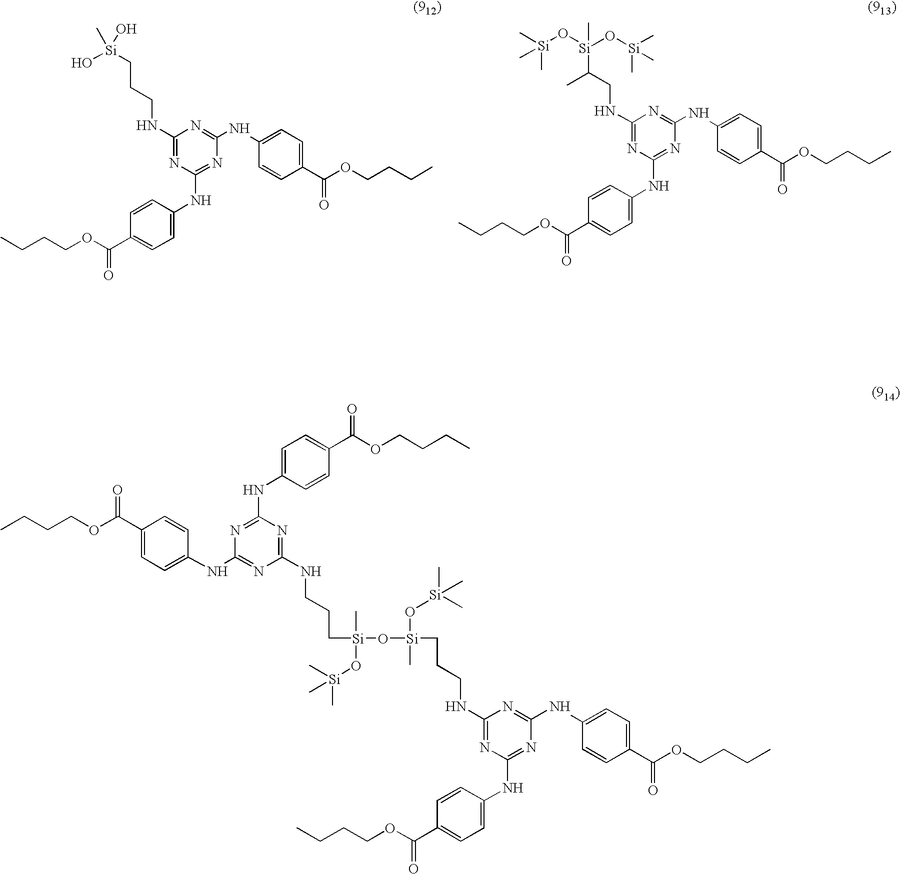

More particularly preferred is the compound 2,4-bis(n-butyl 4′-diylaminobenzoate)-6-{[1,3,3,3-tetramethyl-1-[(trimethylsilyl)oxy]disiloxanyl]propyl-3-ylamino}-s-triazine of structure (9₂):

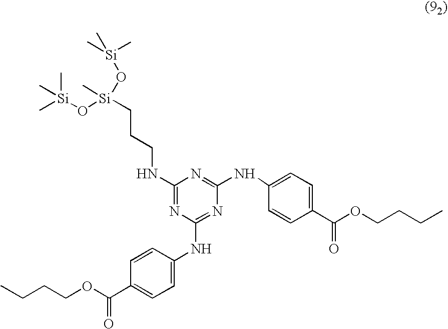

The triazines of formula (9) have been described, as has the synthesis thereof, in EP1891079.

The lipophilic triazines in accordance with the present invention may be selected from among those corresponding to general formula (15) below:

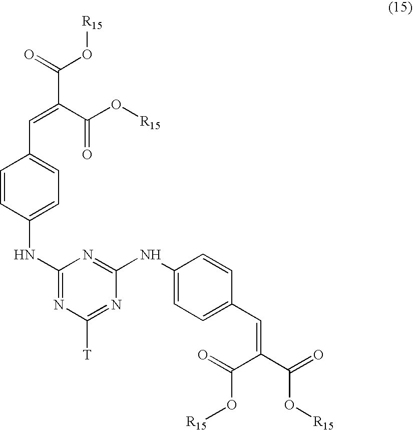

in which:

the radicals R₁₅, which may be identical or different, are each a group of formula (16):

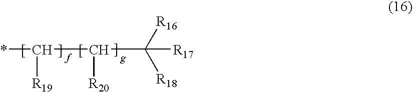

in which:

R₁₆ and R₁₇, which may be identical or different, are each a linear or branched C₁-C₈ alkyl radical;

R₁₆ and R₁₇ may together form a C₅-C₈ ring member optionally substituted with 1, 2 or 3 linear or branched C₁-C₄ alkyl radical(s);

R₁₈, R₁₉ and R₂₀, which may be identical or different, are each a hydrogen atom or a linear or branched C₁-C₄ alkyl radical;

f is 0 or 1;

g is 0 or 1;

with the proviso that:

when f=1 and R₁₉ is hydrogen, then g is equal to 0 and R₁₈ is other than hydrogen,

when R₁₆ and R₁₇ together form a C₅-C₈ ring member, then the sum f+g is other than 2;

T is a divalent radical:

of formula (17) below:

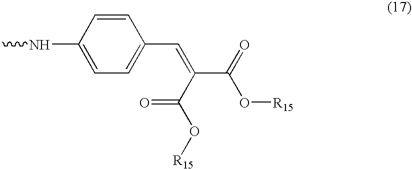

in which R₁₅ has the same definition as in formula (15) above; of formula (18) below:

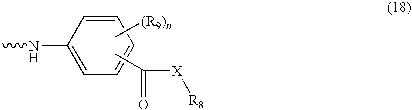

in which R₈, R₉, X and n have the same definition as in formula (9) above;

of formula (19) below:

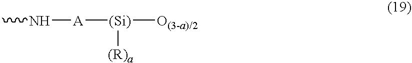

or of formula (20) below:

[symbol]

NH-A-Si(R₁₄)₃ (20)

in which A and R₁₄ have the same definitions as in formula (9c) above.

In addition to the structural units of formula (R)ₐ—(Si)(O)_{(4-a)/2}, the organosiloxane may comprise units of formula: (R)_{b}—(Si)(O)_{(4-b)/2} in which R, a, and A have the same definitions as in formula (I) above and b is 1, 2 or 3.

Among the preferred compounds of formula (15), exemplary are those for which:

f=1 and R₁₉ is a C₁-C₄ alkyl radical and more particularly methyl,

f=1 and R₁₈ is a C₁-C₄ alkyl radical and more particularly methyl,

f=g=0 and R₁₆, R₁₇ and R₁₈ are each a C₁-C₄ alkyl and more particularly methyl,

or else R₁₈ is hydrogen and R₁₆ and R₁₇ together form a C₅-C₈ ring member optionally substituted with 1 or 2 alkyl radicals and more particularly cyclohexyl.

Among the compounds of formula (15) where T is a group of formula (16), which are particularly preferred, exemplary are those selected from among compounds of formulae (15₁) to (15₅) below:

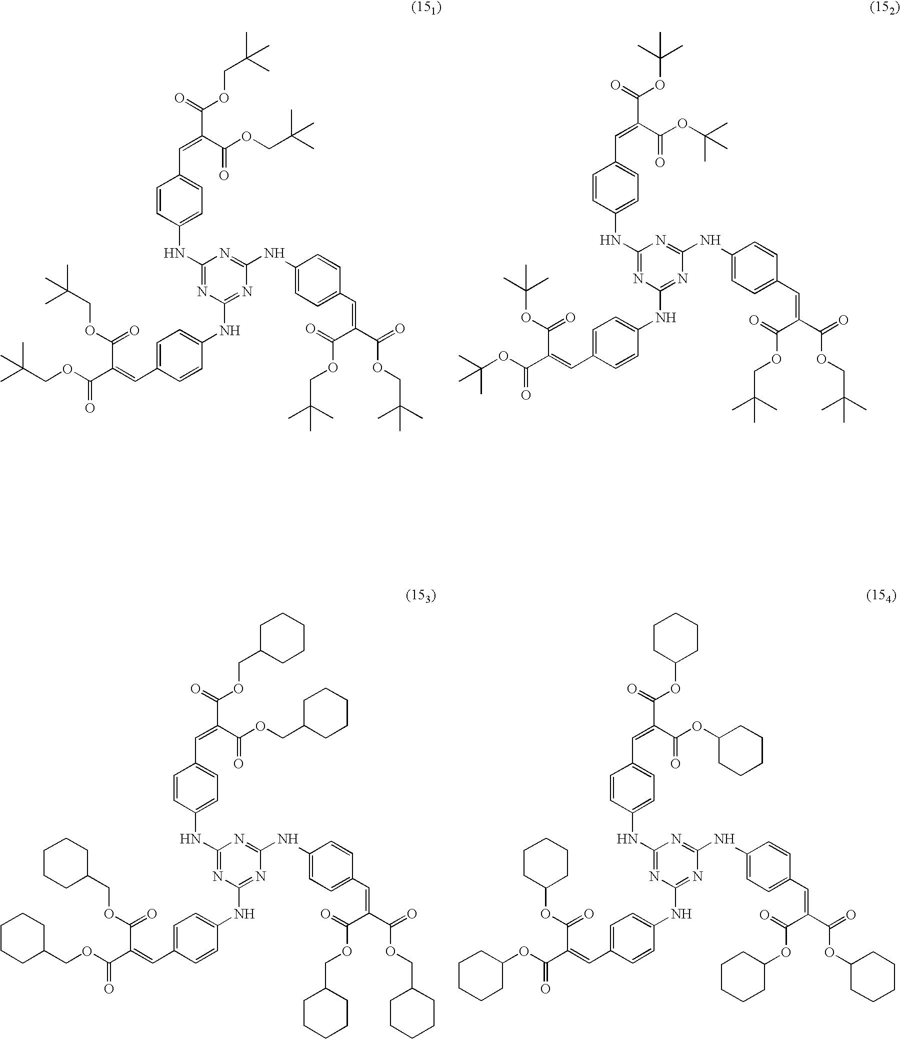

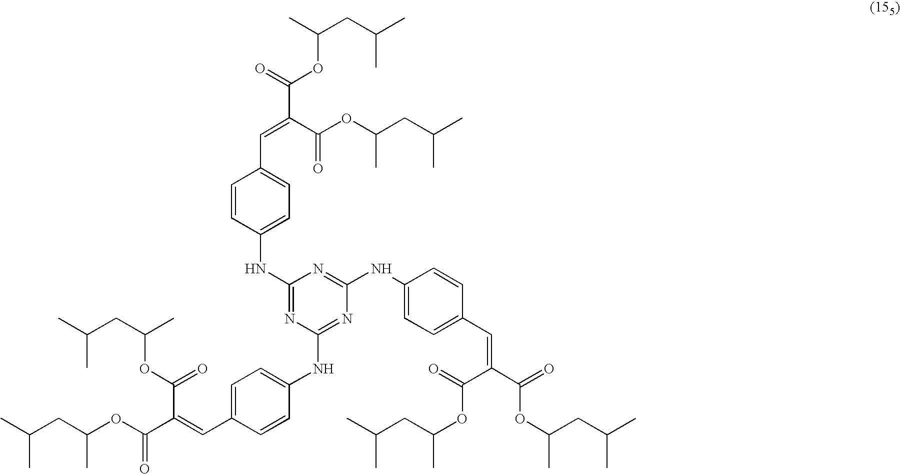

Among the compounds of formula (IX) where T is a group of formula (17), that are particularly preferred, exemplary are those selected from among compounds of formulae (15₆) to (15₁₄) below:

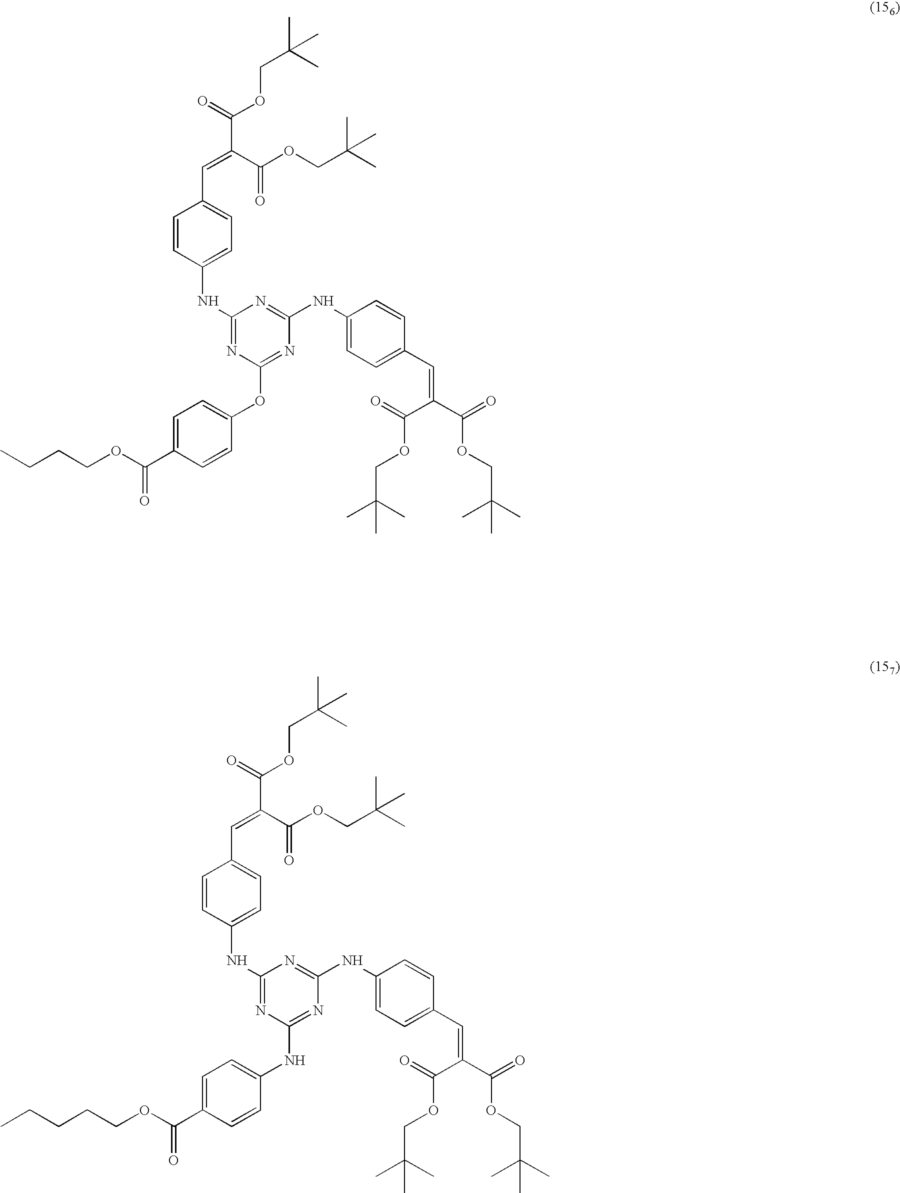

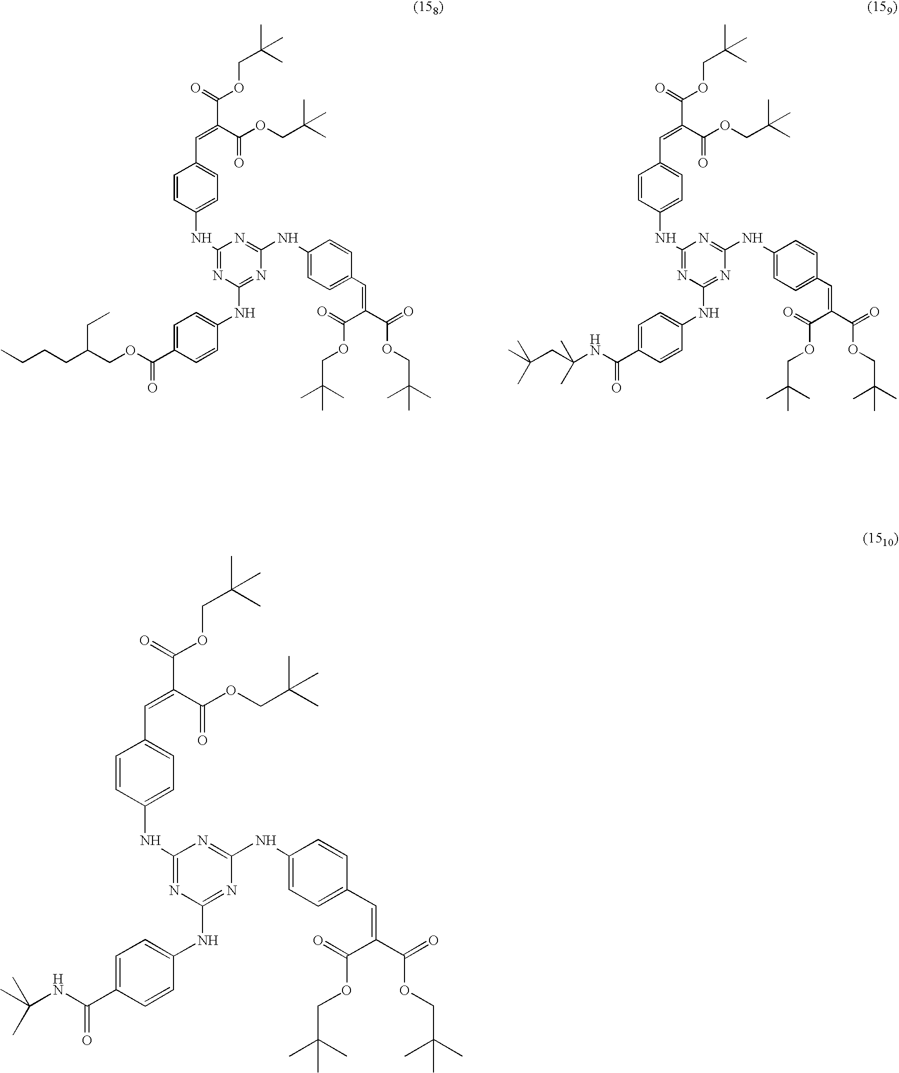

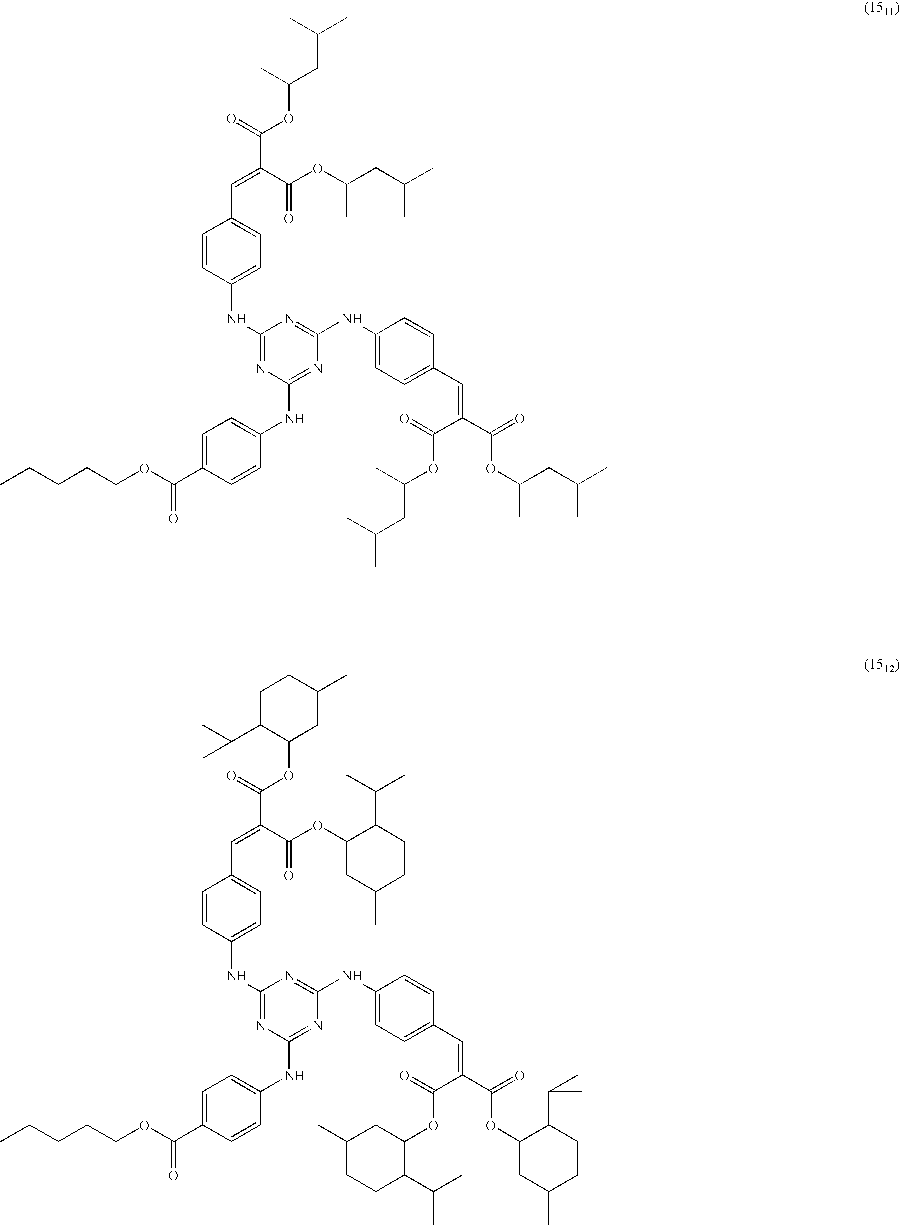

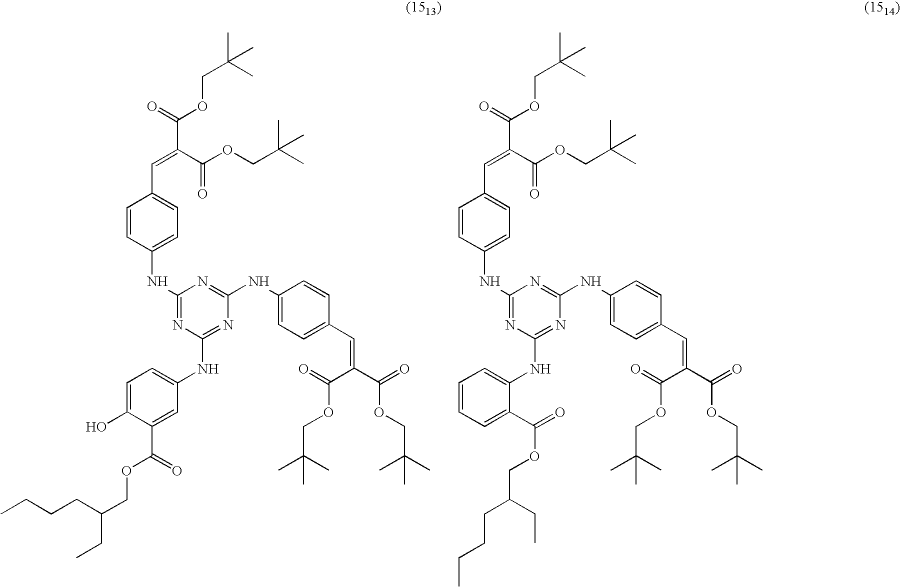

Among the compounds of formula (IX) where T is a group of formula (18) that are particularly preferred, exemplary are those selected from among compounds of formulae (15₁₅) to (15₁₉) below:

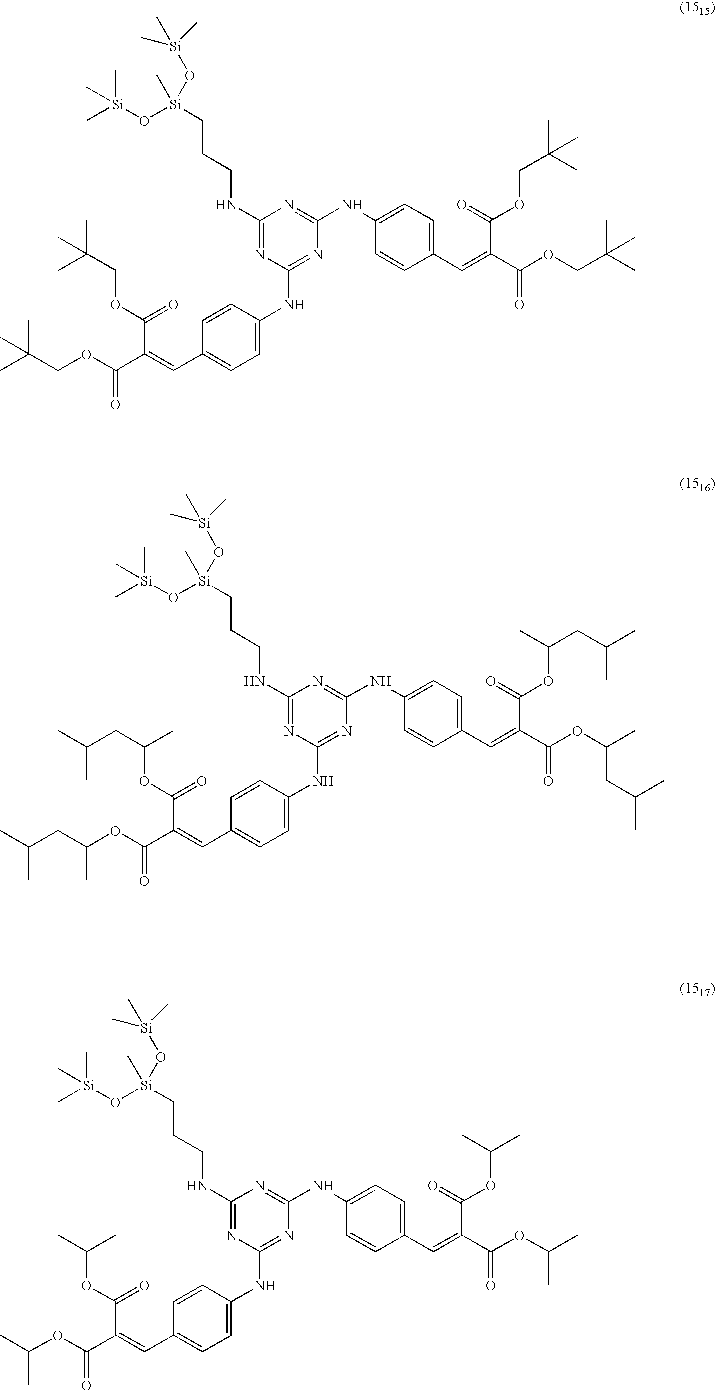

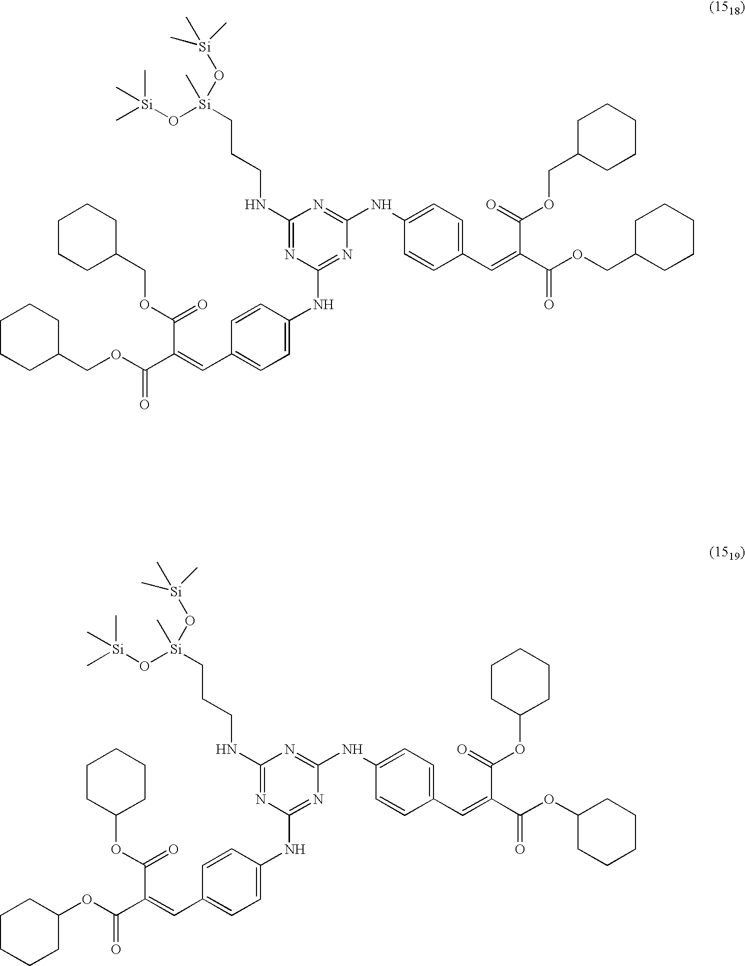

Among the compounds of formula (VII) where T is a group of formula (19) that are particularly preferred, exemplary are those selected from among compounds of formulae (15₂₀) and (15₂₁) below:

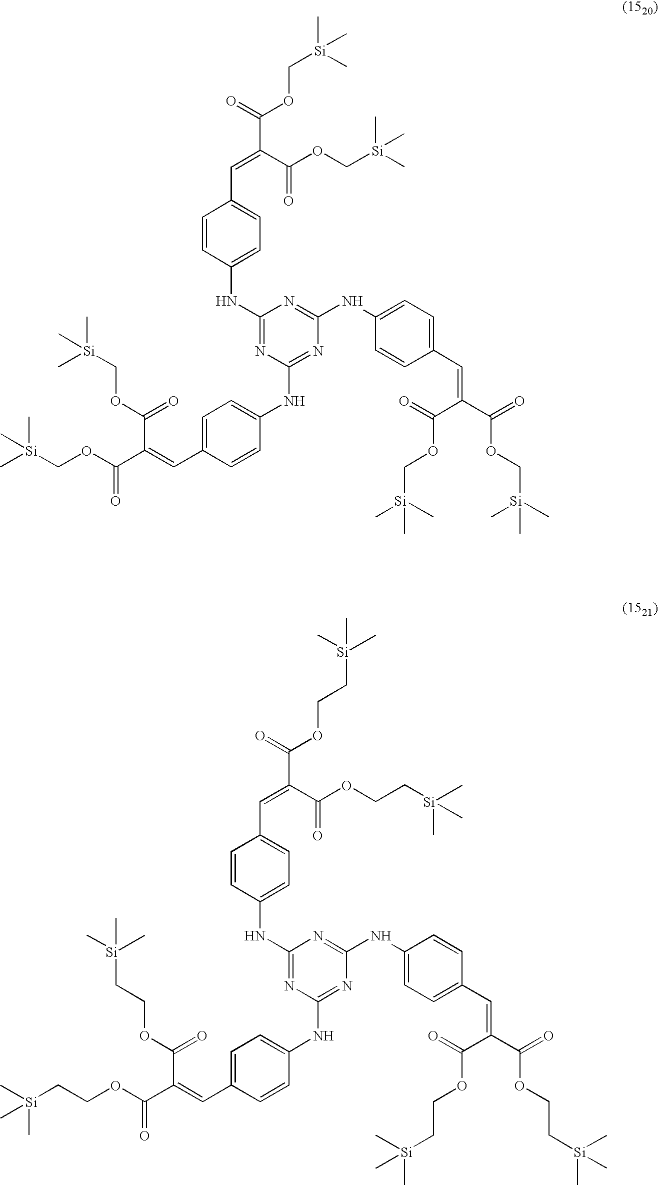

Among these compounds of formula (15), particularly preferred are the compounds (151), (155), (156), (1510), (1515), (1517) and (1520).

Among these compounds, more particularly preferred are the dineopentyl 2-[4-({4,6-bis[(4-{3-(neopentyloxy)-2-[(neopentyloxy)carbonyl]-3-oxoprop-1-enyl}phenyl)amino]-1,3,5-triazin-2-yl}amino)benzylidene]malonate of formula (15₁):

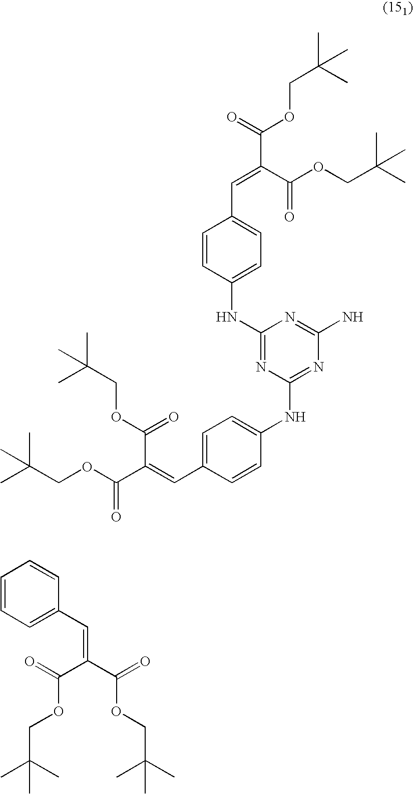

and the 2,4-bis(dineopentyl 4′-aminobenzalmalonate)-6-(butyl 4″-aminobenzoate)-s-triazine of formula (15₆):

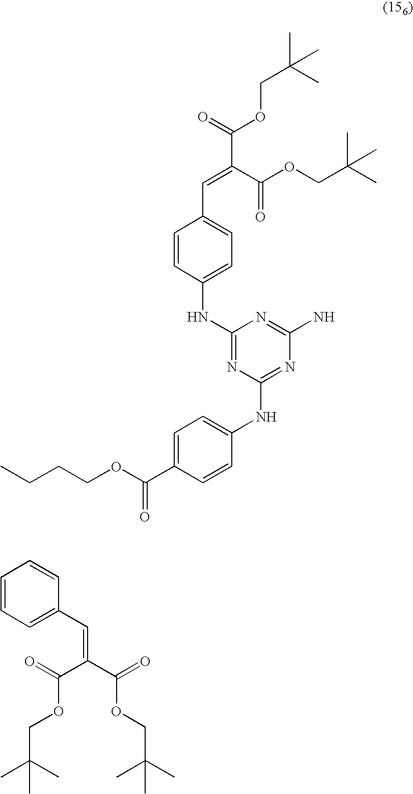

Other examples of triazine-type lipophilic screening agents are the benzoxazole derivatives as described in EP0832642, EP1027883, EP1300137 and DE10162844, such as 2,4-bis[5-1(dimethylpropyl)benzoxazol-2-yl(4-phenyl)imino]-6-(2-ethylhexyl)imino-1,3,5-triazine marketed under the trademark Uvasorb K2A by Sigma 3V.

According to one particular embodiment of the invention, the derivative(s) of formula (I) is (are) present in an amount sufficient to solubilize, by itself or by themselves (without it being necessary to use another solvent), the total amount of lipophilic active agent(s) present in the composition.

The triazine UV-screening agents in accordance with the invention are preferably present in the subject compositions at contents of from 0.01% to 20% by weight, and more preferably from 0.1% to 10%, and even more preferably from 0.1% to 6% by weight, relative to the total weight of the composition.

The compositions of the invention may also contain other additional UV-screening agents that are active in the UV-A and/or UV-B range.

The additional organic screening agents are in particular selected from among anthranilates; dibenzoylmethane derivatives; cinnamic derivatives; salicylic derivatives; camphor derivatives; benzophenone derivatives; β,β-diphenylacrylate derivatives; triazine derivatives that are water-soluble or insoluble in oils and in water; benzotriazole derivatives; benzalmalonate derivatives, in particular those mentioned in U.S. Pat. No. 5,624,663; benzimidazole derivatives; imidazolines; bisbenzoazolyl derivatives as described in EP-669,323 and U.S. Pat. No. 2,463,264; p-aminobenzoic acid (PABA) derivatives;

methylenebis(hydroxyphenylbenzotriazole) derivatives as described in U.S. Pat. Nos. 5,237,071, 5,166,355, GB2303549, DE-197,26,184 and EP893119; screening polymers and screening silicones such as those described in particular in WO-93/04665; dimers derived from α-alkylstyrene, such as those described in DE-198,55,649; 4,4-diarylbutadienes as described in EP0967200, DE-197,46,654, DE-197,55,649, EP-A-1008586, EP-1133980 and EP-133981; merocyanins as described in WO 2004/006878 and WO 2006/003094, the document IP COM Journal 4 (4), 16 No. IPCOM000011179D published on Apr. 3, 2004, and EP-1965870, EP-1962786 and EP-1965869, and mixtures thereof.

Exemplary additional organic photoprotective agents are those noted below under their INCI names:

Dibenzoylmethane Derivatives:

Butyl methoxydibenzoylmethane marketed in particular under the trademark “PARSOL 1789” by Hoffmann Laroche,
Isopropyl dibenzoylmethane.

Cinnamic Derivatives:

Ethylhexyl methoxycinnamate marketed in particular under the trademark Parsol MCX by DSM Nutritional Products, Inc.,
Isopropyl methoxycinnamate,
Isoamyl methoxycinnamate marketed under the trademark Neo Heliopan E 1000 by Symrise,
DEA methoxycinnamate,
Diisopropyl methylcinnamate,
Glyceryl ethylhexanoate dimethoxycinnamate.

para-Aminobenzoic Acid Derivatives:

### PABA,

### Ethyl PABA,

Ethyl dihydroxypropyl PABA,
Ethylhexyl dimethyl PABA marketed in particular under the trademark “Escalol 507” by ISP,

### Glyceryl PABA,

PEG-25 PABA marketed under the trademark “Uvinul P25” by BASF.

Salicylic Derivatives:

Homosalate marketed under the trademark “Eusolex HMS” by Rona/EM Industries,
Ethylhexyl salicylate marketed under the trademark “Neo Heliopan OS” by Symrise,
Dipropylene glycol salicylate marketed under the trademark “Dipsal” by Scher,
TEA salicylate, marketed under the trademark “Neo Heliopan TS” by Symrise.

β,β-Diphenylacrylate Derivatives:

Octocrylene marketed in particular under the trademark “Uvinul N539” by BASF,
Etocrylene, marketed in particular under the trademark “Uvinul N35” by BASF.

Benzophenone Derivatives:

Benzophenone-1 marketed under the trademark “Uvinul 400” by BASF,
Benzophenone-2 marketed under the trademark “Uvinul D50” by BASF,
Benzophenone-3 or oxybenzone, marketed under the trademark “Uvinul M40” by BASF,
Benzophenone-4 marketed under the trademark “Uvinul MS40” by BASF, Benzophenone-5,
Benzophenone-6 marketed under the trademark “Helisorb 11” by Norquay,
Benzophenone-8 marketed under the trademark “Spectra-Sorb UV-24” by American Cyanamid,
Benzophenone-9 marketed under the trademark “Uvinul DS-49” by BASF, Benzophenone-12,
n-Hexyl 2-(4-diethylamino-2-hydroxybenzoyl)benzoate marketed under the trademark “Uvinul A+” by BASF.

Benzylidenecamphor Derivatives:

3-Benzylidenecamphor manufactured under the trademark “Mexoryl SD” by Chimex,
4-Methylbenzylidenecamphor marketed under the trademark “EUSOLEX 6300” by Merck,
Benzylidenecamphorsulfonic acid manufactured under the trademark “Mexoryl SL” by Chimex,
Camphor benzalkonium methosulfate manufactured under the trademark “Mexoryl SO” by Chimex,
Terephthalylidenedicamphorsulfonic acid manufactured under the trademark “Mexoryl SX” by Chimex,
Polyacrylamidomethylbenzylidenecamphor manufactured under the trademark “Mexoryl SW” by Chimex.

Phenylbenzimidazole Derivatives:

Phenylbenzimidazolesulfonic acid marketed in particular under the trademark “EUSOLEX 232” by Merck,
Disodium phenyl dibenzimidazole tetrasulfonate marketed under the trademark “Neo Heliopan AP” by Symrise.

Phenylbenzotriazole Derivatives:

Drometrizole trisiloxane marketed under the trademark “Silatrizole” by Rhodia Chimie,
Methylenebis(benzotriazolyl)tetramethylbutylphenol, marketed in solid form under the trademark “Mixxim BB/100” by Fairmount Chemical, or in micronised form as an aqueous dispersion under the trademark “Tinosorb M” by CIBA Specialty Chemicals.

Anthranilic Derivatives:

Menthyl anthranilate marketed under the trademark “Neo Heliopan MA” by Haarmann and Reimer.

Imidazoline Derivatives:

Ethylhexyldimethoxybenzylidenedioxoimidazoline propionate.

Insoluble Triazine Screening Agents:

The symmetrical triazine screening agents described in U.S. Pat. No. 6,225,467, WO 2004/085412 (see compounds 6 and 9) or in the document “Symetrical Triazine Derivatives” IP.COM Journal, IP.COM INC West Henrietta, N.Y., US (20 Sep. 2004), especially 2,4,6-tris(biphenyl)-1,3,5-triazines (in particular 2,4,6-tris(biphenyl)-4-yl-1,3,5-triazine) and 2,4,6-tris(terphenyl)-1,3,5-triazine which is also noted in WO 06/035000, WO 06/034982, WO 06/034991, WO 06/035007, WO 2006/034992 and WO 2006/034985.

Benzalmalonate Derivatives:

Polyorganosiloxane containing benzalmalonate functions, for instance Polysilicone-15, marketed under the trademark “Parsol SLX” by DSM Nutritional Products, Inc.

4,4-Diarylbutadiene Derivatives:

1,1-Dicarboxy (2,2′-dimethylpropyl)-4,4-diphenylbutadiene.

The preferred additional organic screening agents are selected from among:

Butyl methoxydibenzoylmethane,

Ethylhexyl methoxycinnamate,

Ethylhexyl salicylate,

Homosalate,

Octocrylene,

Phenylbenzimidazolesulfonic acid,

Benzophenone-3,

n-Hexyl 2-(4-diethylamino-2-hydroxybenzoyl)benzoate,

Terephthalylidenedicamphorsulfonic acid,
Disodium phenyl dibenzimidazole tetrasulfonate,

Methylenebis(benzotriazolyl)tetramethylbutylphenol,

2,4,6-tris(Biphenyl)-4-yl-1,3,5-triazine,

Drometrizole trisiloxane,

Polysilicone-15,

Octyl-5-N,N-diethylamino-2-phenylsulfonyl-2,4-pentadienoate, and mixtures thereof.

The additional organic screening agents in accordance with the invention represent, in general, from 0.1% to 30%, preferably from 1% to 25%, of the total weight of the composition.

Of course, one skilled in the art will take care to select the optional additional screening agent(s) and/or the amounts thereof in such a way that the advantageous properties intrinsically associated with the compositions in accordance with the invention are not, or not substantially, impaired by the envisaged addition(s).

The compositions according to the invention may also contain artificial skin tanning and/or browning agents (self-tanning agents), and more particularly dihydroxyacetone (DHA). These are preferably present in amounts ranging from 0.1% to 10% by weight relative to the total weight of the composition.

The aqueous compositions in accordance with the present invention may also comprise conventional cosmetic adjuvants selected in particular from among fatty substances, organic solvents, ionic or non-ionic, hydrophilic or lipophilic thickeners, demulcents, humectants, opacifiers, stabilizers, emollients, silicones, antifoams, fragrances, preservatives, anionic, cationic, non-ionic, zwitterionic or amphoteric surfactants, active agents, fillers, polymers, propellants and basifying or acidifying agents, or any other ingredient normally used in the cosmetics and/or dermatological field.

Of course, one skilled in the art will take care to select the optional additive(s) and/or the amounts thereof in such a way that the advantageous properties intrinsically associated with the compositions in accordance with the invention are not, or not substantially, impaired by the envisaged addition(s).

The fatty substances may be constituted of an oil or a wax other than the apolar waxes as defined previously or mixtures thereof. The term “oil” means a compound that is liquid at ambient temperature. The term “wax” means a compound that is solid or substantially solid at ambient temperature, and the melting point of which is generally above 35° C.

As oils, exemplary are mineral oils (paraffin); plant oils (sweet almond oil, macadamia oil, blackcurrant seed oil, jojoba oil); synthetic oils such as perhydrosqualene, alcohols, fatty amides (such as isopropyl lauroyl sarcosinate marketed under the trademark “Eldew SL-205” by Ajinomoto), fatty acids or esters such as the C₁₂-C₁₅ alkyl benzoate marketed under the trademark “Finsolv TN” or “Witconol TN” by Witco, 2-ethylphenyl benzoate such as the commercial product marketed under the trademark X-Tend 226® by ISP, octyl palmitate, isopropyl lanolate, triglycerides, including those of capric/caprylic acid, dicaprylyl carbonate marketed under the trademark “Cetiol CC” by Cognis, oxyethylenated or oxypropylenated fatty esters and ethers; silicone oils (cyclomethicone, polydimethylsiloxanes or PDMSs) or fluoro oils, and polyalkylenes.

As waxy compounds, exemplary are carnauba wax, beeswax, hydrogenated castor oil, polyethylene waxes and polymethylene waxes, such as that marketed under the trademark Cirebelle 303 by Sasol.

Among the organic solvents, exemplary are lower alcohols and polyols. The latter may be selected from among glycols and glycol ethers such as ethylene glycol, propylene glycol, butylene glycol, dipropylene glycol or diethylene glycol.

As hydrophilic thickeners, exemplary are carboxyvinyl polymers such as Carbopols (Carbomers) and Pemulens (acrylate/C₁₀-C₃₀ alkyl acrylate copolymer); polyacrylamides such as, for example, the crosslinked copolymers marketed under the trademarks Sepigel 305 (C.T.F.A. name: polyacrylamide/C₁₃₋₁₄ isoparaffin/Laureth 7) or Simulgel 600 (C.T.F.A. name: acrylamide/sodium acryloyldimethyltaurate copolymer/isohexadecane/polysorbate 80) by Seppic; polymers and copolymers of 2-acrylamido-2-methylpropanesulfonic acid, optionally crosslinked and/or neutralized, such as the poly(2-acrylamido-2-methylpropanesulfonic acid) marketed by Hoechst under the trademark “Hostacerin AMPS” (CTFA name: ammonium polyacryloyldimethyl taurate) or Simulgel 800 marketed by Seppic (CTFA name: sodium polyacryolyldimethyl taurate/polysorbate 80/sorbitan oleate); copolymers of 2-acrylamido-2-methylpropanesulfonic acid and of hydroxyethyl acrylate, such as Simulgel NS and Sepinov EMT 10 marketed by Seppic; cellulose derivatives such as hydroxyethylcellulose; polysaccharides and especially gums such as xanthan gum; and mixtures thereof.

As lipophilic thickeners, exemplary are synthetic polymers such as poly(C₁₀-C₃₀ alkyl acrylates) marketed under the trademark “Intelimer IPA 13-1” and “Intelimer IPA 13-6” by Landec, or else modified clays such as hectorite and derivatives thereof, for instance the products marketed under the trademark Bentone.

Among the active agents, exemplary are:

vitamins (C, K, PP, etc.) and derivatives or precursors thereof, alone or as mixtures;

agents for combating pollution and/or free-radical scavengers;

depigmenting agents and/or propigmenting agents;

anti-glycation agents;

calmatives;

NO-synthase inhibitors;

agents for stimulating the synthesis of dermal or epidermal macromolecules and/or for preventing their degradation;

agents for stimulating fibroblast proliferation;

gents for stimulating keratinocyte proliferation;

myorelaxants;

tensioning agents;

matting agents;

keratolytic agents;

desquamating agents;

moisturizers;

anti-inflammatory agents;

agents that act on the energy metabolism of cells;

insect repellents;

substance P antagonists and CRGP antagonists;

agents for preventing hair loss and/or for hair regrowth; and

anti-wrinkle agents.

The compositions according to the invention can be formulated according to techniques well known to those skilled in the art. They may in particular be in the form of a simple or complex emulsion (O/W, W/O, O/W/O or W/O/W) such as a cream, a milk or a cream gel; in the form of an aqueous gel; or in the form of a lotion. They may optionally be packed in an aerosol and be in the form of a foam or a spray.

Preferably, the compositions according to the invention are in the form of an oil-in-water or water-in-oil emulsion.

The emulsions generally contain at least one emulsifier selected from among amphoteric, anionic, cationic or non-ionic emulsifiers, employed alone or as a mixture. The emulsifiers are selected in an appropriate manner according to the emulsion to be obtained (W/O or O/W). The emulsions may also contain other types of stabilizers, for instance fillers or gelling or thickening polymers.

As emulsifying surfactants that can be used for preparing W/O emulsions, exemplary are, for example, of alkyl esters or ethers of sorbitan, of glycerol or of sugars; silicone surfactants, for instance dimethicone copolyols, such as the mixture of cyclomethicone and of dimethicone copolyol marketed under the trademark “DC 5225 C” by Dow Corning, and alkyl dimethicone copolyols, such as the lauryl methicone copolyol marketed under the trademark “Dow Corning 5200 Formulation Aid” by Dow Corning; cetyl dimethicone copolyol, such as the product marketed under the trademark Abil EM 90R by Goldschmidt and the mixture of cetyl dimethicone copolyol, of polyglycerol isostearate (4 mol) and of hexyl laurate marketed under the trademark Abil WE 09 by Goldschmidt. It is possible to also add thereto one or more coemulsifiers, which advantageously may be selected from among group comprising alkylated polyol esters.

Exemplary alkylated polyol esters are polyethylene glycol esters, for instance PEG-30 dipolyhydroxystearate, such as the product marketed under the trademark Arlacel P135 by ICI.

As glycerol and/or sorbitan esters, exemplary are polyglyceryl isostearate, such as the product marketed under the trademark Isolan GI 34 by Goldschmidt; sorbitan isostearate, such as the product marketed under the trademark Arlacel 987 by ICI; sorbitan glyceryl isostearate, such as the product marketed under the trademark Arlacel 986 by ICI, and mixtures thereof.

For the O/W emulsions, exemplary emulsifiers are, non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters such as the PEG-100 stearate/glyceryl stearate mixture marketed, for example, by ICI under the trademark Arlacel 165; oxyalkylenated (oxyethylenated and/or oxypropylenated) fatty alcohol ethers; sugar esters such as sucrose stearate; ethers of a fatty alcohol and of a sugar, in particular alkylpolyglucosides (APGs), such as decylglucoside and laurylglucoside marketed, for example, by Henkel under the respective names Plantaren 2000 and Plantaren 1200, cetostearyl glucoside optionally as a mixture with cetostearyl alcohol, marketed, for example, under the trademark Montanov 68 by Seppic, under the trademark Tegocare CG90 by Goldschmidt and under the trademark Emulgade KE3302 by Henkel, and also arachidyl glucoside, for example in the form of the mixture of arachidyl and behenyl alcohols and arachidyl glucoside marketed under the trademark Montanov 202 by Seppic. According to one particular embodiment of the invention, the mixture of alkylpolyglucoside as defined above with the corresponding fatty alcohol may be in the form of a self-emulsifying composition, as described, for example, in WO-A-92/06778.

Other emulsion stabilizers more particularly exemplary are polymers of isophthalic acid or of sulfoisophthalic acid, and in particular phthalate/sulfoisophthalate/glycol copolymers, for example the diethylene glycol/phthalate/isophthalate/1,4-cyclohexanedimethanol copolymer (INCI name: Polyester-5) marketed under the trademarks “Eastman AQ polymer” (AQ35S, AQ38S, AQ55S, AQ48 Ultra) by Eastman Chemical.

When an emulsion is provided, the aqueous phase of said emulsion may comprise a non-ionic vesicular dispersion prepared according to known methods (Bangham, Standish and Watkins. J. Mol. Biol., 3, 238 (1965), FR-2,315,991 and FR-2,416,008).

The compositions according to the invention find applications in a large number of treatments, in particular cosmetic treatments, for the skin, the lips and the hair, including the scalp, in particular for protecting and/or caring for the skin, the lips and/or the hair, and/or for making up the skin and/or the lips.

The present invention also features formulation of the subject compositions into products for the cosmetic treatment of the skin, the lips, the nails, the hair, the eyelashes, the eyebrows and/or the scalp, in particular care products, sunscreen products and makeup products.

The cosmetic compositions according to the invention may, for example, be used as a makeup product.

The cosmetic compositions according to the invention may, for example, be used as a care product and/or sunscreen product for the face and/or the body, of liquid to semi-liquid consistency, such as milks, more or less rich creams, cream gels, or pastes. They may optionally be packaged in an aerosol and may be in the form of a foam or a spray.

The compositions according to the invention in the form of vaporizable fluid lotions in accordance with the invention are applied to the skin or the hair in the form of fine particles using pressurized devices. The devices in accordance with the invention are well known to those skilled in the art and comprise non-aerosol pump-dispensers or “atomizers”, aerosol containers comprising a propellant and also aerosol pump-dispensers that use compressed air as a propellant. The latter are described in U.S. Pat. Nos. 4,077,441 and 4,850,517.

The compositions packaged as an aerosol in accordance with the invention generally contain conventional propellants such as, for example, hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

In order to further illustrate the present invention and the advantages thereof, the following specific examples are given, it being understood that same are intended only as illustrative and in nowise imitative. In said examples to follow, all parts and percentages are given by weight, unless otherwise indicated.

Solubility of Lipophilic Triazine Screening Agents, Compared with Prior Art Solvents and the Solvents of the Invention:

Screening Agents Tested:

Screening agent 1: Dineopentyl 2-[4-({4,6-bis[(4-{3-(neopentyloxy)-2-[(neopentyloxy)carbonyl]-3-oxoprop-1-enyl}phenyl)amino]-1,3,5-triazin-2-yl}amino)benzylidene]malonate

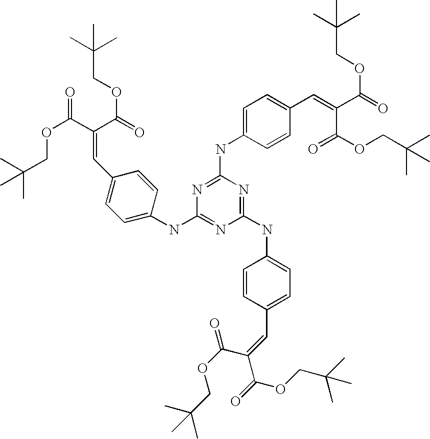

Screening agent 2: 2,4-bis(isobutyl 4′-aminobenzalmalonate)-6-[(3-{1,3,3,3-tetramethyl-1-[(trimethylsilyl)oxy]disiloxanyl}propyl)amino]-1,3,5-triazine=example 5 of EP-0841341:

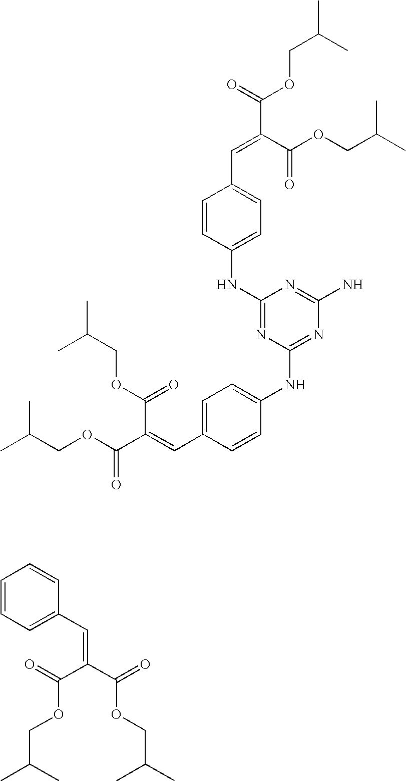

Screening agent 3: Dineopentyl 2-[4-({4-{[4-(butoxycarbonyl)phenyl]amino}-6-[(4-{3-(neopentyloxy)-2-[(neopentyloxy)carbonyl]-3-oxoprop-1-enyl}phenyl)amino]-1,3,5-triazin-2-yl}amino)benzylidene]malonate

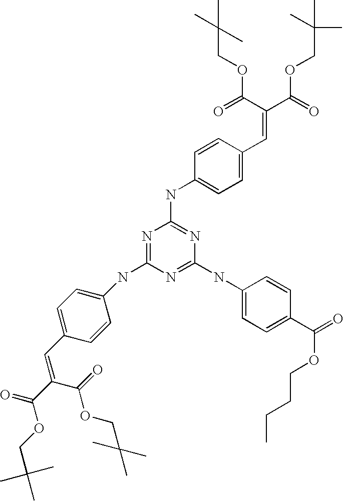

Screening agent 4: Dineopentyl 2-[4-({4-({4-[(tert-butylamino)carbonyl]phenyl}amino)-6-[(4-{3-(neopentyloxy)-2-[(neopentyloxy)carbonyl]-3-oxoprop-1-enyl}phenyl)amino]-1,3,5-triazin-2-yl}amino)benzylidene]malonate

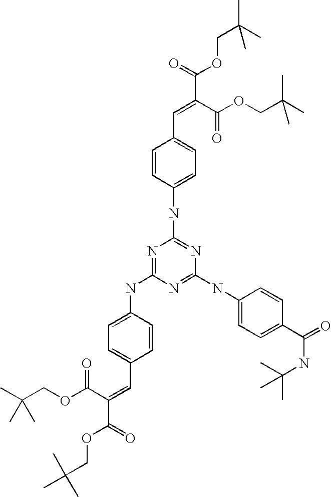

Screening agent 5: N-(1,1,3,3-tetramethylbutyl)-4-({4-[(4-{[(1,1,3,3-tetramethylbutyl)amino]carbonyl}phenyl)amino]-6-[(3-{1,3,3,3-tetramethyl-1-[(trimethylsilyl)oxy]disiloxanyl}propyl)amino]-1,3,5-triazin-2-yl}amino)benzamide

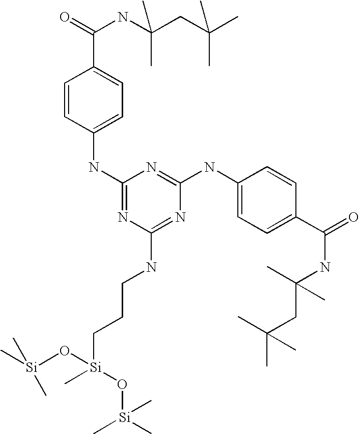

Screening agent 6: Ethyl 4-({4-{[4-(ethoxycarbonyl)phenyl]amino}-6-[(3-{1,3,3,3-tetramethyl-1-[(trimethylsily)oxy]disiloxanyl}propyl)amino]-1,3,5-triazin-2-yl}amino)benzoate

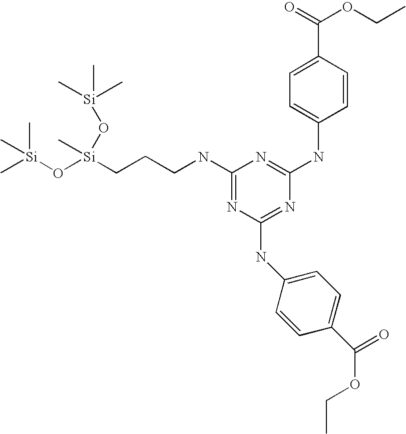

Screening agent 7: 2-Ethylhexyl 4-({4-({4-[(tert-butylamino)carbonyl]phenyl}amino)-6-[(4-{[(2-ethylhexyl)oxy]carbonyl}phenyl)amino]-1,3,5-triazin-2-yl}amino)benzoate

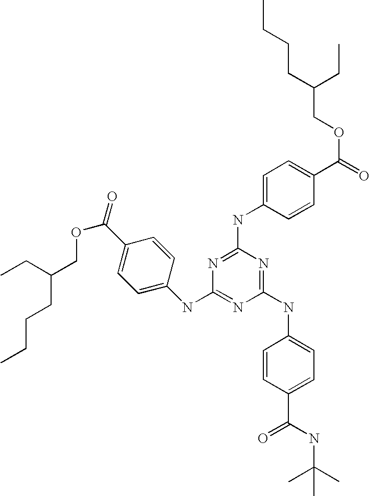

Screening agent 8: Butyl 4-({4-{[4-(butoxycarbonyl)phenyl]amino}-6-[(3-{1,3,3,3-tetramethyl-1-[(trimethylsilyl)oxy]disiloxanyl}propyl)amino]-1,3,5-triazin-2-yl}amino)benzoate

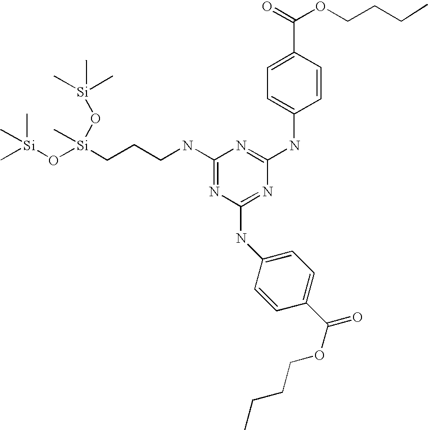

Screening agent 9: 2-Ethylhexyl 4-({4-({4-[(tert-butylamino)carbonyl]phenyl}amino)-6-[(4-{[(2-ethylhexyl)oxy]carbonyl}phenyl)amino]-1,3,5-triazin-2-yl}amino)benzoate (known under the trademark Uvinul T150 from BASF):

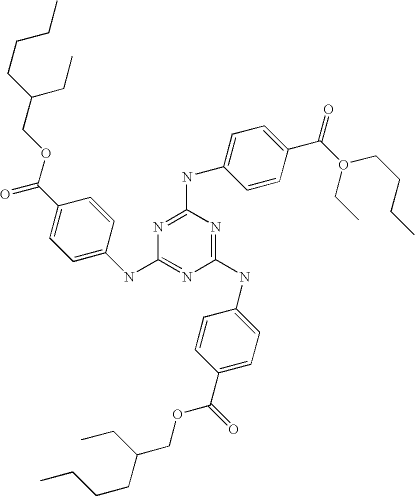

Screening agent 10: 5-[(2-Ethylhexyl)oxy]-2-[4-{4-[(2-ethylhexyl)oxy]-2-hydroxyphenyl}-6-(4-methoxyphenyl)-1,3,5-triazin-2-yl]phenol (known under the trademark Tinosorb S from Ciba):

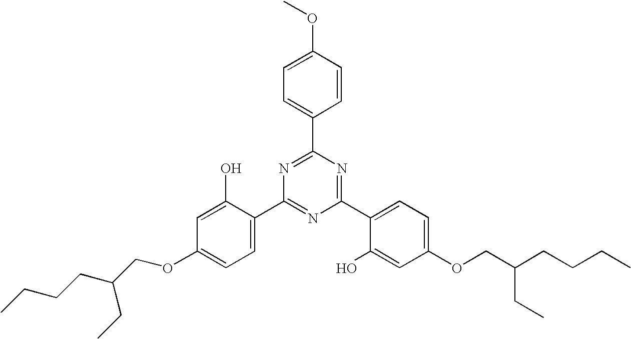

Comparative oil 1: Isopropyl N-lauroylsarcosinate: Eldew SL-205 from Ajinomoto, of formula:

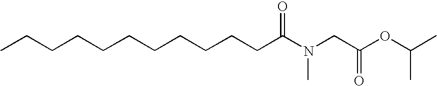

Comparative oil 2: Finsolv TN: C₁₂-C₁₅ alkyl benzoate

Comparative oil 3: Miglyol 812: Caprylic/capric acid triglycerides

Comparative oil 4: X-Tend: 2-Phenylethyl benzoate

Procedure:

X mg of product are introduced into Y mg of oil; with mild heating (<60° C.) and the use of a sonicator for 1 minute, the solution obtained is left at laboratory temperature for 1 month; the state of this solution is observed; if no crystal or oily deposit is visible, the solubility of the product is considered to be greater than X×100/(X+Y) weight/weight; when crystals or an oily deposit appear, the test is repeated with 5% less product.

TABLE 1

| | | Compound |
|---|---|---|
| | Isopropyl N- | (m) of the |
| Triazine screening agent | lauroylsarcosinate | invention |
| Screening agent 1 | 40% gel | 60% gel |
| Screening agent 3 | 40% gel | 60% gel |
| Screening agent 4 | 40% gel | 60% gel |
| Screening agent 5 | 40% gel | 60% gel |
| Screening agent 7 | 40% gel | 60% gel |

TABLE 2

| Oil tested | Screening agent 2 |
|---|---|
| Isopropyl N-lauroylsarcosinate | 35% |
| C₁₂-C₁₅ alkyl benzoate | 8% |
| Caprylic/capric acid triglycerides | 6% |
| Compound (m) of the invention | 60% gel |

TABLE 3

| Oil tested | Screening agent 6 |
|---|---|
| Isopropyl N-lauroylsarcosinate | 40% gel |
| C₁₂-C₁₅ alkyl benzoate | 40% gel |
| Caprylic/capric acid triglycerides | 40% gel |
| 2-Phenylethyl benzoate | 40% gel |
| Compound (m) of the invention | 60% gel |

TABLE 4

| Oil tested | Screening agent 7 |
|---|---|
| C₁₂-C₁₅ alkyl benzoate | 40% gel |
| Compound (m) of the invention | 60% gel |

TABLE 5

| Oil tested | Screening agent 8 |
|---|---|
| Isopropyl N-lauroylsarcosinate | 40% gel |
| C₁₂-C₁₅ alkyl benzoate | 28% |
| Caprylic/capric acid triglycerides | 31% |
| 2-Phenylethyl benzoate | 27% |
| Compound (m) of the invention | 60% gel |
| Compound (j) of the invention | 60% gel |
| Compound (I) of the invention | 60% gel |
| Compound (ii) of the invention | 60% gel |

TABLE 6

| Oil tested | Screening agent 9 |
|---|---|
| Isopropyl N-lauroylsarcosinate | 40% |
| C₁₂-C₁₅ alkyl benzoate | 5% |
| Compound (m) of the invention | 60% gel |

TABLE 7

| Oil tested | Screening agent 10 |
|---|---|
| Isopropyl N-lauroylsarcosinate | 10% |
| Compound (m) of the invention | 18% |
| Compound (v) of the invention | 20% |

### Formulation Examples 1-4

The following formulations were prepared, the amounts are expressed as percentages by weight relative to the total weight of the composition.

| | | | | |
|---|---|---|---|---|
| Composition | Ex 1 | Ex 2 | Ex 3 | Ex 4 |

Phase A

| | | | | |
|---|---|---|---|---|
| Polydimethylsiloxane | 0.5 | 0.5 | 0.5 | 0.5 |
| Preservatives | 1 | 1 | 1 | 1 |
| Stearic acid | 1.5 | 1.5 | 1.5 | 1.5 |
| Glyceryl monostearate/ | 1 | 1 | 1 | 1 |
| PEG100 stearate mixture | | | | |
| Mixture of cetylstearyl glucoside | 2 | 2 | 2 | 2 |
| and of cetyl, stearyl alcohols | | | | |
| Cetyl alcohol | 0.5 | 0.5 | 0.5 | 0.5 |
| Butyl 1-butyl-5-oxopyrrolidine-3- | 20 | | 15 | — |
| carboxylate (compound (m)) | | | | |
| Methyl 1-ethyl-5-oxopyrrolidine-3- | | 20 | | 15 |
| carboxylate (compound (v)) | | | | |
| Bis{ethylhexyloxy-2- | 5 | 5 | | |
| hydroxyphenyl}-6- | | | | |
| (methoxyphenyl)-1,3,5-triazine | | | | |
| (Tinosorb S-CIBA) | | | | |
| 2,4,6-Tris[ethylhexyl- | — | — | 5 | 5 |
| oxycarbonyl)anilino]-1,3,5-triazine | | | | |
| (Uvinul T150-BASF) | | | | |

Phase B

| | | | | |
|---|---|---|---|---|
| Deionized water | qs 100 | qs 100 | qs 100 | qs 100 |
| Glycerol | 5 | 5 | 5 | 5 |
| Xanthan gum | 0.2 | 0.2 | 0.2 | 0.2 |
| Monocetyl phosphate | 1 | 1 | 1 | 1 |

Phase C

| | | | | |
|---|---|---|---|---|
| Isohexadecane | 1 | 1 | 1 | 1 |
| Acrylic acid/stearyl methacrylate | 0.2 | 0.2 | 0.2 | 0.2 |
| copolymer | | | | |
| Triethanolamine | qs | qs | qs | qs |
| | pH | pH | pH | pH |

Procedure:

The aqueous phase (Phase B) containing all its ingredients is heated to 80° C. in a waterbath. The fatty phase (Phase A) containing all its ingredients is heated to 80° C. in a waterbath. A is emulsified in B with rotor-stator stirring (instrument from the company Moritz). Phase C is incorporated and the resulting mixture is left to return to ambient temperature with moderate stirring. The triethanolamine is introduced so as to adjust the pH to the desired value at the end of production.

The anti-sun emulsions obtained are stable with respect to storage and do not display any crystals or precipitates.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable UV-screening composition comprising at least one ester solvent derived from 4-carboxy-2-pyrrolidinone and having the formula (I) below: 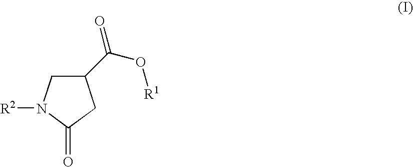
in which:
R¹ is a linear or branched C₁-C₂₀ alkyl radical, and
R² is a linear or branched C₁-C₂₀ alkyl radical optionally comprising a C₅-C₆ ring member, the phenyl radical, the benzyl radical or the phenethyl radical, and at least one triazine-type lipophilic UV-screening agent, formulated into a topically applicable, cosmetically acceptable medium therefor.

2. The UV-screening composition as defined by claim 1, in which the at least one ester solvent derived from 4-carboxy-2-pyrrolidinone, of formula (I), is selected from among the following compounds:

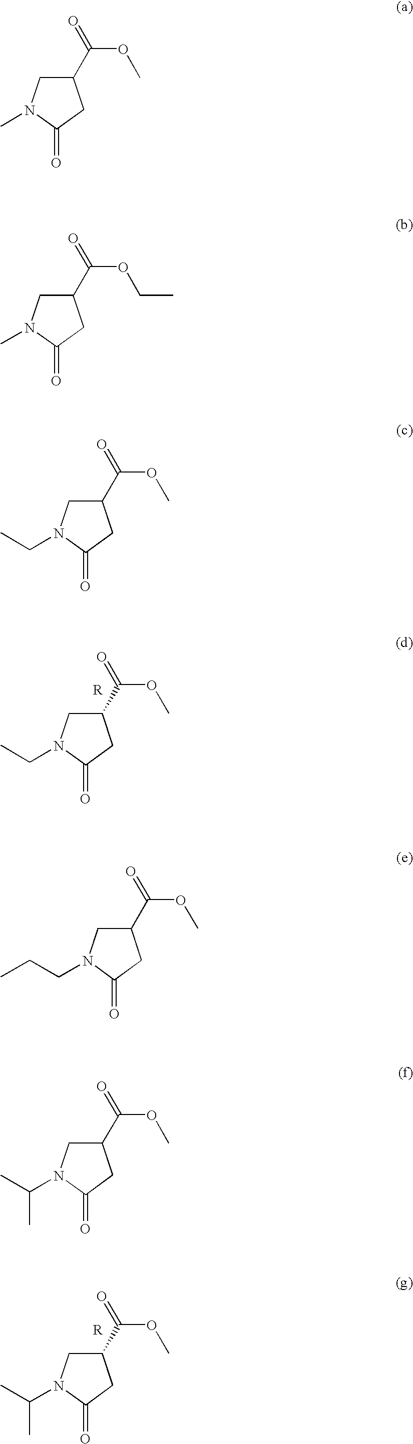

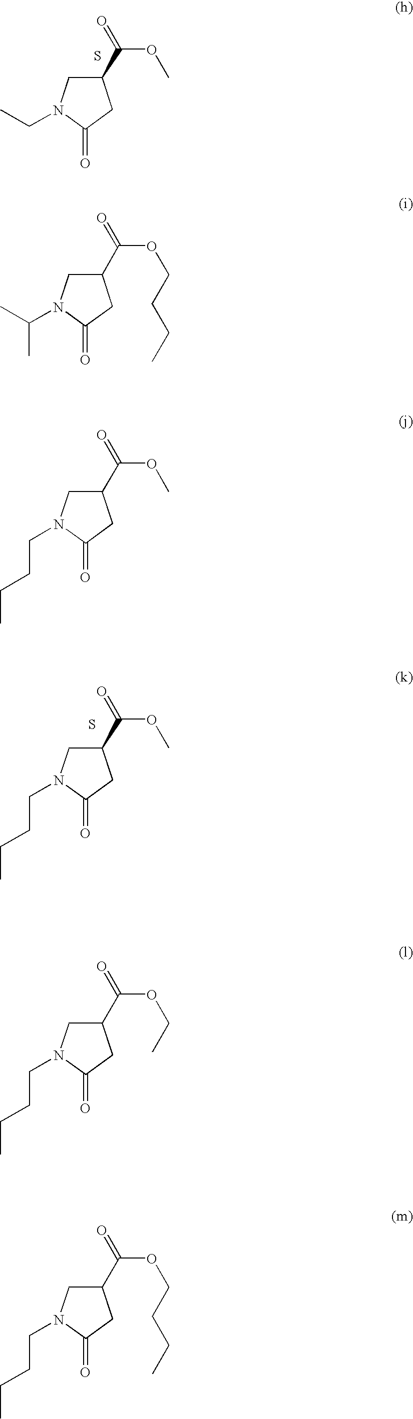

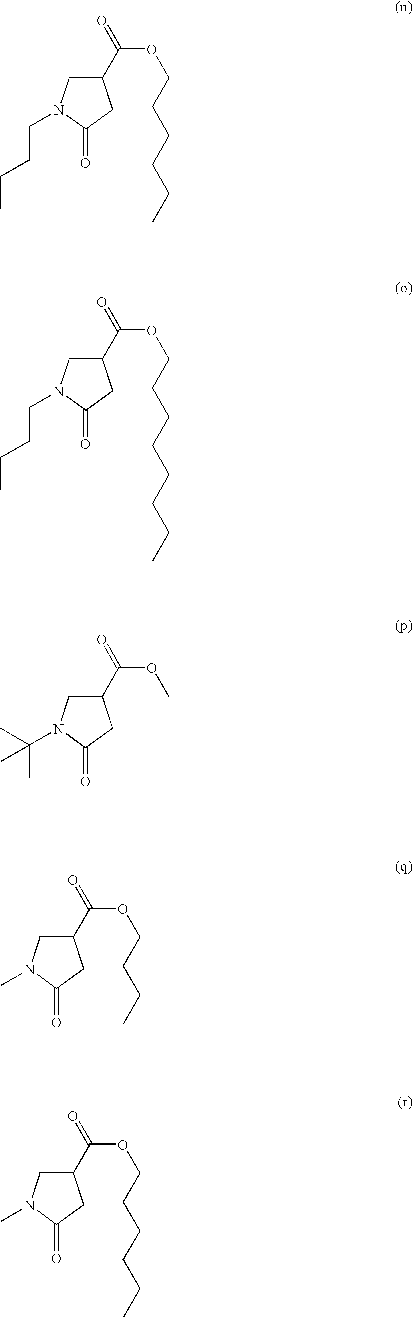

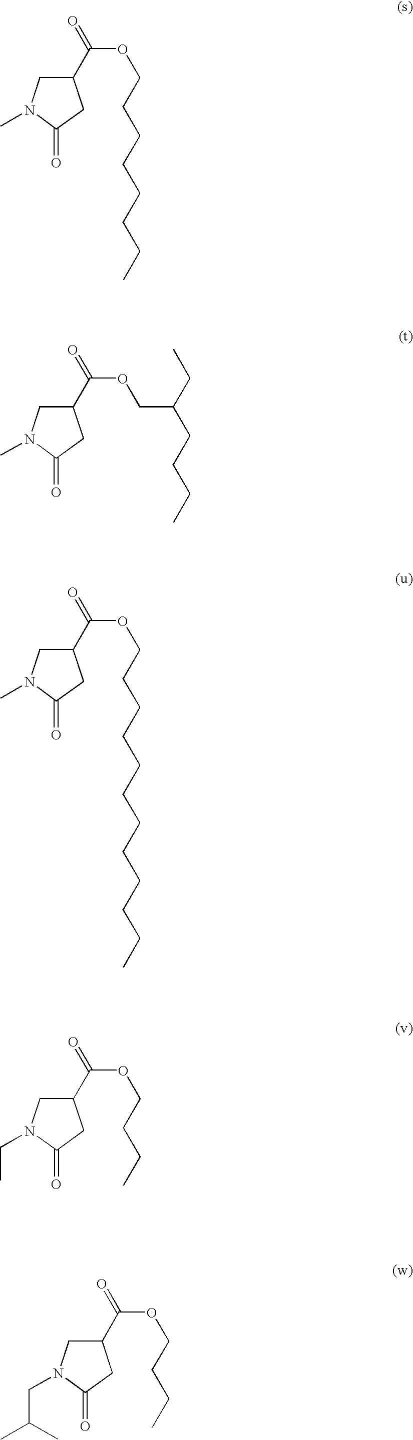

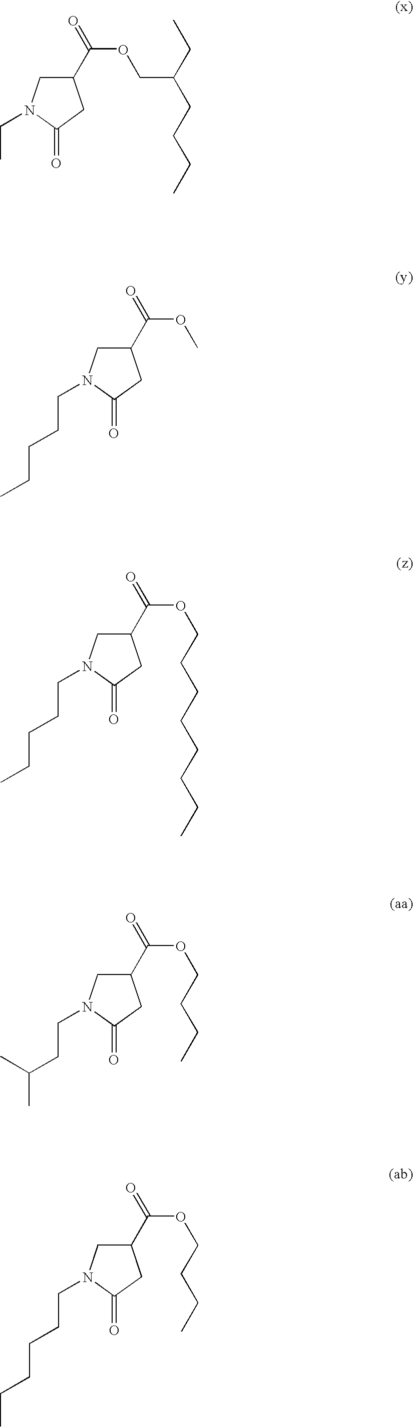

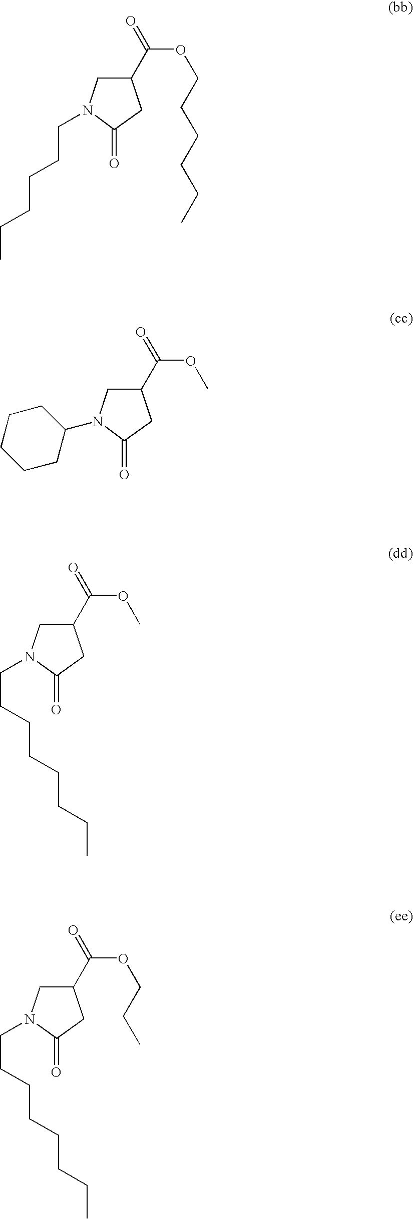

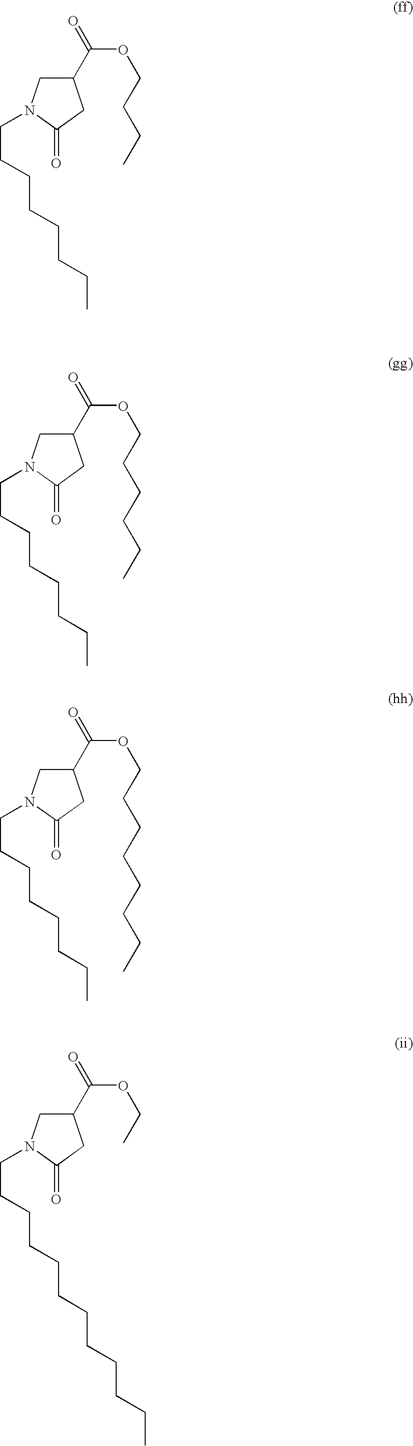

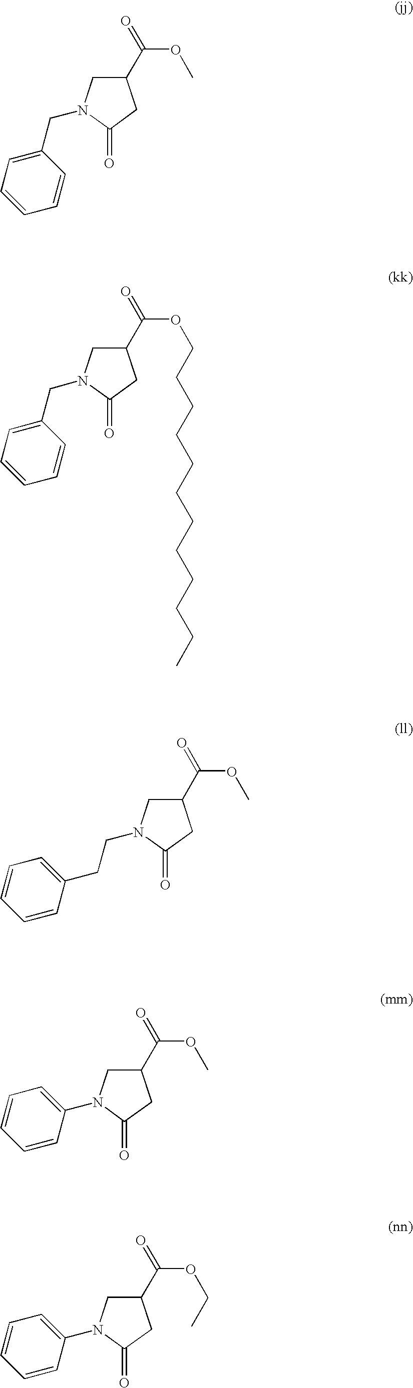

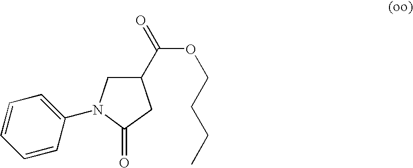

3. The UV-screening composition as defined by claim 2, in which the compounds of formula (I) are selected from among the compounds (t), (u), (v), (x), (j), (l), (m), (w), (n), (ab), (ii) and (kk).

4. The UV-screening composition as defined by claim 1, in which the at least one triazine-type UV-screening agent is selected from among the compounds of formula (1) below: 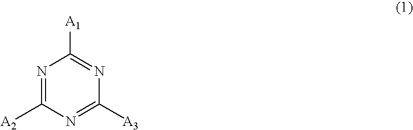
in which the A₁, A₂ and A₃ radicals, which may be identical or different, are each selected from among radicals of formula (2): 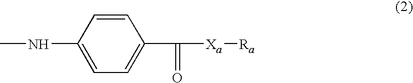
in which:
the bridges Xₐ which may be identical or different, are each oxygen or an —NH— radical;
the radicals Rₐ, which may be identical or different, are each selected from among: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylenated radical comprising from 1 to 6 ethylene oxide units and the terminal OH group of which is methylated; a radical of formula (3), (4) or (5) below: 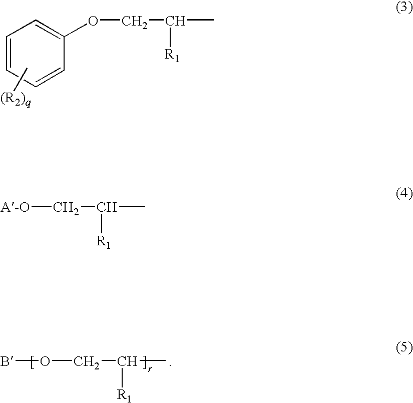
in which:
R₁ is hydrogen or a methyl radical;
R₂ is a C₁-C₉ alkyl radical;
g is an integer ranging from 0 to 3;
r is an integer ranging from 1 to 10;
A′ is a C₄-C₈ alkyl radical or a C₅-C₈ cycloalkyl radical;
B′ is selected from among: a linear or branched C₁-C₈ alkyl radical; a C₅-C₈ cycloalkyl radical; and an aryl radical optionally substituted with one or more C₁-C₄ alkyl radicals.

5. The UV-screening composition according to claim 4, in which the at least one triazine screening agent of formula (I) is selected from among:
2-[(p-(tert-butylamido)anilino]-4,6-bis[(p-(2′-ethylhexyl-1′-oxycarbonyl)anilino]-1,3,5-triazine corresponding to the formula below: 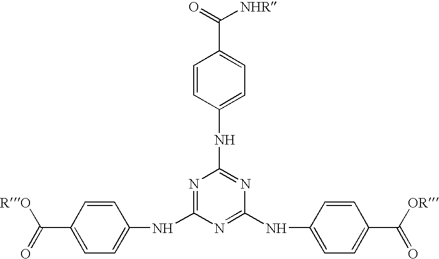
2,4,6-tris[p-(2′-ethylhexyl-1′-oxycarbonyl)anilino]-1,3,5-triazine having the formula below: 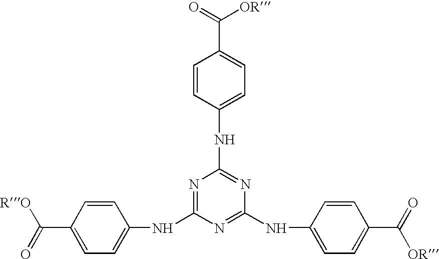
in which R′″ is a 2-ethylhexyl radical and R″ is a tert-butyl radical.

6. The UV-screening composition as defined by claim 1, in which the at least one triazine screening agent is selected from among compounds of formula (6) below: 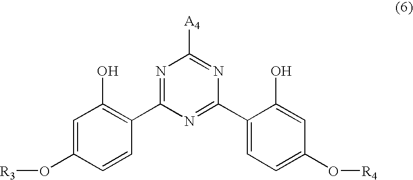
in which:
the R₃ and R₄ radicals, which may be identical or different, are each a C₃-C₁₈ alkyl radical, a C₂-C₁₈ alkenyl radical or a residue of formula —CH₂—CH(OH)—CH₂—OT₁ wherein T₁ is a hydrogen atom or a C₁-C₈ alkyl radical;
A₄ is a residue having one of the formulae below: 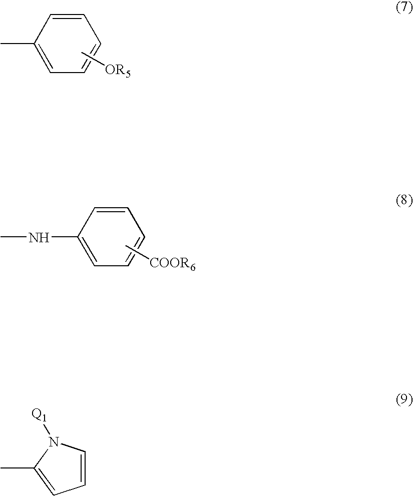
in which:
R₅ is a hydrogen atom, a C₁-C₁₀ alkyl radical, a radical of formula: —(CH₂CHR₇—O)ₙ₁R₆ wherein n₁ is a number from 1 to 16, or else a residue having the structure —CH₂—CH—(OH)—CH₂OT₁ with T₁ having the same definition as indicated above;
R₆ is hydrogen, a metal cation M, a C₁-C₅ alkyl radical or a residue of formula —(CH₂)ₘ₂—OT₁ where m₂ is a number from 1 to 4 and T₁ has the same definition as indicated above;
R₇ is hydrogen or methyl; and
Q₁ is a C₁-C₁₈ alkyl radical.

7. The UV-screening composition as defined by claim 6, in which the triazine of formula (6) comprises 2,4-bis{[4-(2-ethylhexyloxy)-2-hydroxy]phenyl}-6-(4-methoxyphenyl)-1,3,5-triazine.

8. The UV-screening composition as defined by claim 1, in which the at least one triazine screening agent is selected from among:
2,4,6-tris(diisobutyl 4′-aminobenzalmalonate)-6-butoxy-s-triazine,
2,4,6-tris(diisobutyl 4′-aminobenzalmalonate)-6-(2-ethylhexylamino)-s-triazine,
2,4-bis(4′-aminobenzylidenecamphor)-6-(2-ethylhexylamino)-s-triazine,
2,4,6-bis(4′ aminobenzylidenecamphor)-6-(diisobutyl 4′-aminobenzalmalonate)-s-triazine,
2,4,6-tris(diethyl 4′-aminobenzalmalonate)-s-triazine,
2,4,6-tris(diisopropyl 4′-aminobenzalmalonate)-s-triazine,
2,4,6-tris(diisobutyl 4′-aminobenzalmalonate)-s-triazine,
2,4,6-tris(dineopentyl 4′-aminobenzalmalonate)-s-triazine,
2,4-bis(n-butyl 4′-aminobenzoate)-6-(aminopropyltrisiloxane)-s-triazine.

9. The UV-screening composition as defined by claim 1, in which the at least one triazine screening agent is selected from among compounds of general formula (9) below, or a tautomeric form thereof: 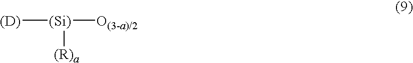
in which:
the radicals R, which may be identical or different, are each a linear or branched and optionally halogenated or unsaturated C₁-C₃₀ alkyl radical, a C₆-C₁₂ aryl radical, a C₁-C₁₀ alkoxy radical, a hydroxyl radical or the trimethylsilyloxy group;
a=1 to 3; and in addition to structural units of formula -A-(Si)(R)ₐ(O)_{(3-a)/2}, the organosiloxane optionally comprises structural units of formula: (R)_{b}—(Si)(O)_{(4-b)/2} with b=1, 2 or 3;
the group (D) is an s-triazine compound of formula (10) below: 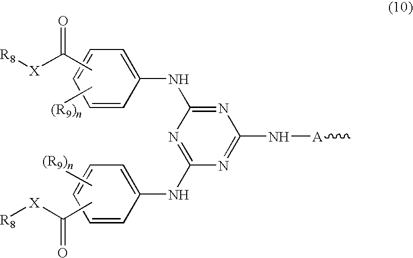
wherein:
X is —O— or —NR₁₀—, in which R₁₀ is hydrogen or a C₁-C₅ alkyl radical,
R₈ is a linear or branched, optionally unsaturated, C₁-C₃₀ alkyl radical optionally containing a silicon atom, a C₅-C₂₀ cycloalkyl radical optionally substituted with 1 to 3 linear or branched C₁-C₄ alkyl radicals, the —(CH₂CHR₁₀—O)ₘR₁₁ radical or the —CH₂—CH(OH)—CH₂—O—R₁₂ radical,
the radicals R₉, which may be identical or different, are each a hydroxyl radical, a linear or branched C₁-C₈ alkyl radical, or a C₁-C₈ alkoxy radical, with the proviso that two adjacent R₂ radicals of the same aromatic ring may together form an alkylidene dioxy group in which the alkylidene radical contains 1 or 2 carbon atoms,
R₁₀ is hydrogen or methyl; with the proviso that the (C═O)XR₈ group to be in the ortho-, meta- or para-position with respect to the amino group,
R₁₁ is hydrogen or a C₁-C₈ alkyl radical,
R₁₂ is hydrogen or a C₄-C₈ alkyl radical,
m is an integer ranging from 2 to 20,
n=0 to 2,
A is a divalent radical selected from among methylene or a radical corresponding to one of formula (11), (12), (13) or (14) below: 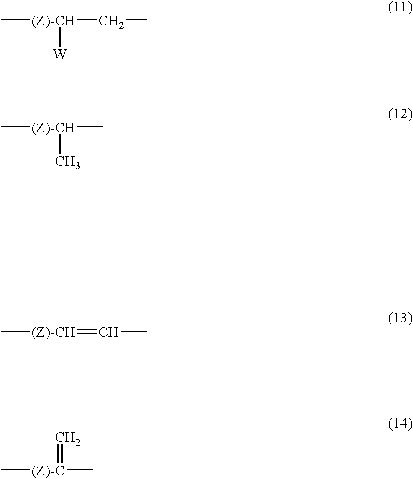
in which:
Z is a saturated or unsaturated, linear or branched C₁-C₁₀ alkylene diradical optionally substituted with a hydroxyl radical or oxygens and may optionally contain an amino group, and
W is a hydrogen atom, a hydroxyl radical or a saturated or unsaturated, linear or branched C₁-C₈ alkyl radical.

10. The UV-screening composition as defined by claim 9, in which the triazine of formula (9) comprises the 2,4-bis(n-butyl 4′-diylaminobenzoate)-6-{[1,3,3,3-tetramethyl-1-[(trimethylsilyl)oxy]disiloxanyl]propyl-3-ylamino}-s-triazine of structure (9₂):

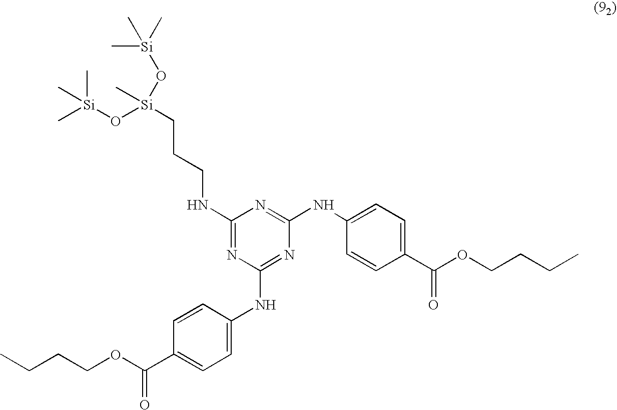

11. The UV-screening composition as defined by claim 1, in which the at least one triazine screening agent is selected from among compounds of general formula (15) below: 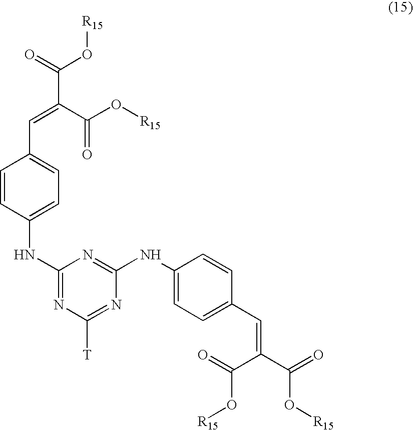
in which:
the radicals R₁₅, which may be identical or different, are each a radical of formula (16): 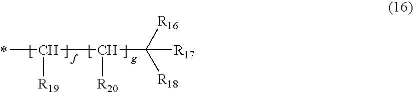
in which:
R₁₆ and R₁₇, which may be identical or different, are each a linear or branched C₁-C₈ alkyl radical;
R₁₆ and R₁₇ may together form a C₅-C₈ ring member optionally substituted with 1, 2 or 3 linear or branched C₁-C₄ alkyl radical(s);
R₁₈, R₁₉ and R₂₀, which may be identical or different, are each a hydrogen atom or a linear or branched C₁-C₄ alkyl radical;
f is 0 or 1;
g is 0 or 1;
with the proviso that:
when f=1 and R₁₉ is hydrogen, then g is equal to 0 and R₁₈ is other than hydrogen,
when R₁₆ and R₁₇ together form a C₅-C₈ ring member, then the sum f+g is other than 2;
T is a divalent radical:
of formula (17) below: 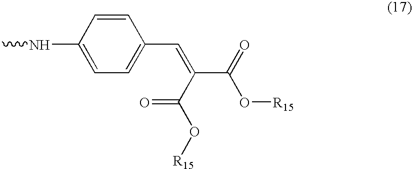
in which R₁₅ has the same definition as in formula (15) above;
of formula (18) below: 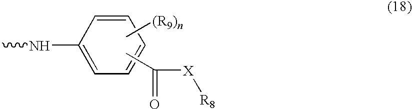
in which R₈, R₉, X and n have the same definition as in formula (9);
of formula (19) below: 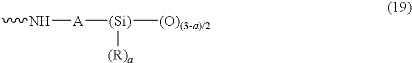
or of formula (20) below: 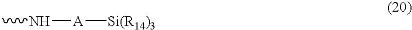
in which A and R₁₄ have the same definitions as in formula (9c).

12. The UV-screening composition as defined by claim 11, in which the triazine of formula (15) is selected from among:
dineopentyl 2-[4-({4,6-bis[(4-{3-(neopentyloxy)-2-[(neopentyloxy)carbonyl]-3-oxoprop-1-enyl}phenyl)amino]-1,3,5-triazin-2-yl}amino)benzylidene]malonate of formula (15₁): 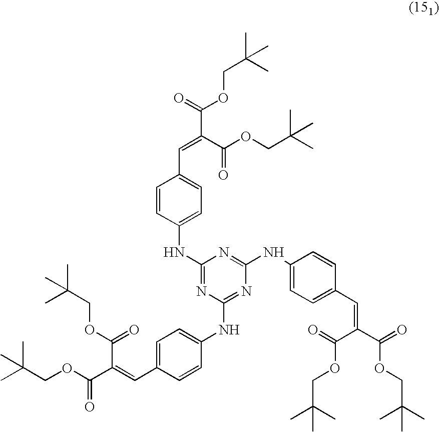
or the 2,4-bis(dineopentyl 4′-aminobenzalmalonate)-6-(butyl 4″-aminobenzoate)-s-triazine of formula (15₆):

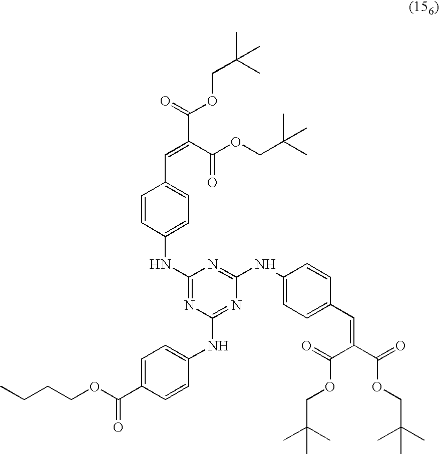

13. The UV-screening composition as defined by claim 1, in which the derivative(s) of formula (I) is (are) present in an amount sufficient to solubilize, by itself or by themselves, the total amount of lipophilic triazine screening agent(s) present in the composition.

14. A method comprising formulating at least one ester derived from 4-carboxy-2-pyrrolidinone, of formula (I), as defined by claim 1, into a composition which comprises, in a cosmetically acceptable medium, at least one triazine-type lipophilic UV-screening agent, as a solvent for said triazine UV-screening agent, in and/or as an agent for improving the solubility of said triazine UV-screening agent in said composition.

15. A regime or regimen for photoprotecting the skin against the damaging effects of UV-radiation, comprising topically applying thereon a thus effective amount of the UV-screening composition as defined by claim 1.
